(19) United States
(12) Patent Application Publication
(10) Pub. No.: US 2025/0262155 A1
(43) Pub. Date: Aug. 21, 2025

(54) LITHE LIPOSOMES COMPRISING ABSORPTION ENHANCERS FOR ORAL DRUG DELIVERY

(71) Applicant: Aspire LLC, Somerset, NJ (US)

(72) Inventors: Zhang Zhang, Somerset, NJ (US); Madhav Pai, Somerset, NJ (US)

(73) Assignee: Aspire LLC, Somerset, NJ (US)

(21) Appl. No.: 19/014,005

(22) Filed: Jan. 8, 2025

Related U.S. Application Data
(60) Provisional application No. 63/618,496, filed on Jan. 8, 2024.

(51) Int. Cl. A61K 9/1271; A61K 31/00; A61K 31/07; A61K 31/12; A61K 31/197; A61K 31/375; A61K 31/455; A61K 31/51; A61K 31/525; A61K 31/593; A61K 31/714

(52) U.S. Cl. CPC A61K 9/1271; A61K 31/07; A61K 31/12; A61K 31/197; A61K 31/375; A61K 31/455; A61K 31/51; A61K 31/525; A61K 31/593; A61K 31/658; A61K 31/714

(57) ABSTRACT

The present disclosure provides lithe liposome compositions capable of improving the oral bioavailability of encapsulated active agents. Such lithe liposomes can comprise a bilayer structure modified by one or more lipid bilayer modifiers and/or one or more absorption enhancers. The present disclosure also provides methods of making and using such lithe liposome compositions.

### RELATED APPLICATIONS

This application claims priority to U.S. Provisional Application No. 63/618,496 filed on Jan. 8, 2024, the contents of which are incorporated herein by reference in their entirety.

### BACKGROUND

Liposomes have important applications in both medicine and dietary supplements as delivery systems, mainly due to their biocompatibility, biodegradability, low toxicity, and capability to encapsulate and protect both hydrophilic and lipophilic actives from degradation and clearance. (Kim et al. Liposomes: Biomedical Applications. Chonnam Med J. 57(1):27-35, 2021).

Despite advances in the application of liposomes as injectable drug delivery systems, the need exists for liposomes to facilitate oral drug delivery.

The key parameters controlling the absorption of active components are the dissolution of the actives in the gastrointestinal (GI) tract and the permeability of the drug through the GI membrane. Therefore, oral bioavailability is dependent on the actives' aqueous solubility, stability and permeability. Actives that are hydrophobic, unstable or with low permeability require a high dose but have low systemic exposure, resulting in potential adverse effects and may vary in their effect in individual consumers.

Furthermore, the delivery of pharmaceutical or dietary ingredients that are susceptible to GI environment is challenging because of the combined detrimental effects of gastric acid, bile salts and pancreatic lipases, as well as poor permeability across intestinal epithelia because of the relatively large size of particles and the presence of various epithelial barriers. Accordingly, there is a need for new liposome drug delivery systems with enhanced stability in the gastrointestinal tract and improved oral absorption of the encapsulated active ingredients.

### SUMMARY

The present disclosure provides compositions and methods for delivering one or more active agent(s) to a subject using a lithe liposome (also referred to herein as a “Vecell”). The compositions can comprise one or more absorption enhancer(s) and/or one or more lipid bilayer modifier(s), which make the liposomes structurally flexible, and improve the solubility and permeability of an encapsulated active agent. In some embodiments, the compositions may comprise one or more absorption enhancer(s) and one or more lipid bilayer modifier(s).

The lithe liposome compositions of the present disclosure provide, inter alia, a controllable means to fine-tune the bioavailability, e.g., the oral bioavailability, of active agents. The lithe liposome compositions are designed to improve the stability, solubility, and permeability of active agents for effective oral delivery. Delivery of an active agent using the lithe liposome compositions described herein can improve the oral bioavailability of the active agent as compared to delivery of the active agent in the absence of the lithe liposome composition, for example, by using a conventional drug delivery system, such as an oil-based composition and/or a micellized composition. Such conventional drug delivery systems can suffer from poor bioavailability and fluctuations in plasma drug level and are often unable to achieve effective oral delivery. Conventional drug delivery systems can also require high dosing and/or dosing frequency, which can be associated with poor patient compliance and adverse effects other than the effects intended by the drug. Without wishing to be bound by a theory, the lithe liposome compositions of the present disclosure can improve the oral bioavailability of an active agent by modulating, e.g., increasing, the stability, solubility, and/or permeability of the active agent.

Accordingly, in a first aspect, the disclosure features a lithe liposome composition, comprising: (i) a phospholipid; (ii) a lipid bilayer modifier, wherein the lipid bilayer modifier is selected from the group consisting of a surfactant, a solvent, a polysaccharide, and a combination thereof; (iii) an absorption enhancer, wherein the absorption enhancer is selected from the group consisting of a terpene, a polyphenol, and a derivative thereof, and a combination thereof; and (iv) an active agent. In some embodiments, the phospholipid is selected from the group consisting of a glycerophospholipid, a sphingophospholipid, a derivative thereof, and a combination thereof.

In some embodiments, the phospholipid is selected from the group consisting of a phosphatidylcholine (PC), a phosphatidylinositol (PI), a phosphatidylinositol phosphate (PIP), a phosphatidylinositol bisphosphate (PIP2), a phosphatidylinositol trisphosphate (PIP3), a phosphorylglycerol (PG), a phosphatidic acid (PA), a phosphatidylethanolamine (PE), a phosphatidylserine (PS), a sphingomyelin (SPM), a sphingosylphosphorylethanolamine (SPE), cardiolipin (CL), a derivative thereof, and a combination thereof. In some embodiments, the phospholipid is selected from the group consisting of a natural phospholipid, a synthetic phospholipid, and a combination thereof.

In some embodiments, the phospholipid comprises a phosphatidylcholine (PC) or a derivative thereof. In some embodiments, the phospholipid is present in an amount of about 0.01% w/v to about 50% w/v, optionally wherein the composition comprises: a phospholipid in an amount of about 0.01% w/v to about 15% w/v; a phospholipid in an amount of about 0.01% w/v to about 10% w/v; a phospholipid in an amount of about 0.01% w/v to about 5% w/v; a phospholipid in an amount of about 0.01% w/v to about 1% w/v; a phospholipid in an amount of about 0.1% w/v to about 1% w/v; a phospholipid in an amount of about 0.5% w/v to about 5% w/v; a phospholipid in an amount of about 5% w/v to about 15% w/v; a phospholipid in an amount of about 10% w/v to about 15% w/v; a phospholipid in an amount of about 1% w/v to about 3% w/v; a phospholipid in an amount of about 2% w/v; or a phospholipid in an amount of about 1% w/v.

In some embodiments, the lipid bilayer modifier is selected from the group consisting of an anionic surfactant, a cationic surfactant, a zwitterionic surfactant, a non-ionic surfactant, and a combination thereof. In some embodiments, the lipid bilayer modifier is selected from the group consisting of a polysorbate 20, a polysorbate 60, and a polysorbate 80, a span 20, a span 40, a span 60, a span 80, a bile salt, sodium cholate, cyclodextrin, ethanol, and a combination thereof. In some embodiments, the lipid bilayer modifier is present in an amount of about 0.01% w/v to about 50% w/v, optionally wherein the composition comprises: a lipid bilayer modifier in an amount of about 0.01% w/v to about 15% w/v; a lipid bilayer modifier in an amount of about 0.01% w/v to about 10% w/v; a lipid bilayer modifier in an amount of about 0.01% w/v to about 5% w/v; a lipid bilayer modifier in an amount of about 0.01% w/v to about 1% w/v; a lipid bilayer modifier in an amount of about 0.1% w/v to about 1% w/v; a lipid bilayer modifier in an amount of about 0.5% w/v to about 5% w/v; a lipid bilayer modifier in an amount of about 5% w/v to about 15% w/v; a lipid bilayer modifier in an amount of about 10% w/v to about 15% w/v; a lipid bilayer modifier in an amount of about 1% w/v to about 3% w/v; a lipid bilayer modifier in an amount of about 2% w/v; or a lipid bilayer modifier in an amount of about 1% w/v.

In some embodiments, the absorption enhancer comprises a terpene. In some embodiments, the terpene is selected from the group consisting of limonene, camphor, menthol, carvone, terpineol, thujone, a derivative thereof, and a combination thereof. In some embodiments, the terpene comprises menthol.

In some embodiments, the absorption enhancer comprises a polyphenol. In some embodiments, the polyphenol comprises a flavonoid. In some embodiments, the flavonoid is selected from the group consisting of an isoflavone, a neoflavonoid, a flavone, a flavonol, a flavanone, a flavanonol, a flavanol, a catechin, an anthocyanin, a chalcone, and a derivative thereof, and a combination thereof. In some embodiments, the flavonoid is selected from the group consisting of cyanidin, malvidin, delphinidin, peonidin, phloretin, arbutin, phlioridzin, chalconaringenin, hesperitin, naringin, naringenin, eriodictyol, hesperidin, apigenin, tangeretin, baicalein, rpoifolin, quercetin, dihydroquercetin, myricetin, rutin, morin, kaempferol, genistin, genistein, daidzein, glycitein, daidzin, a derivative thereof, and a combination thereof. In some embodiments, the composition is substantially free of quercetin and/or dihydroquercetin. In some embodiments, the flavonoid comprises quercetin, dihydroquercetin, or a combination thereof.

In some embodiments, the absorption enhancer comprises a terpene and a polyphenol.

In some embodiments, the absorption enhancer is present in an amount of about 0.01% w/v to about 50% w/v, optionally wherein the composition comprises: an absorption enhancer in an amount of about 0.01% w/v to about 15% w/v; an absorption enhancer in an amount of about 0.01% w/v to about 10% w/v; an absorption enhancer in an amount of about 0.01% w/v to about 5% w/v; an absorption enhancer in an amount of about 0.01% w/v to about 1% w/v; an absorption enhancer in an amount of about 0.1% w/v to about 1% w/v; an absorption enhancer in an amount of about 0.5% w/v to about 5% w/v; an absorption enhancer in an amount of about 5% w/v to about 15% w/v; an absorption enhancer in an amount of about 10% w/v to about 15% w/v; an absorption enhancer in an amount of about 1% w/v to about 3% w/v; an absorption enhancer in an amount of about 2% w/v; or an absorption enhancer in an amount of about 1% w/v.

In some embodiments, the active agent is characterized by one or more of the following: (i) low solubility and high permeability; (ii) high solubility and low permeability; (iii) low solubility and low permeability; (iv) low bioavailability; (v) moderate to severe side effects; and (iv) instability; and combinations thereof. In some embodiments, the active agent is characterized according to a biopharmaceutical classification system (BCS) as set forth in Table 1, optionally wherein the active agent is selected from the group consisting of (i) a Class I drug, optionally characterized by an apparent permeability (Papp) of greater than about 10⁻⁵ (cm/sec) and a dose/solubility (Q) of less than or equal to about 0.5; (ii) a Class II drug, optionally characterized by an Papp of greater than about 10⁻⁵ (cm/sec) and a Q of greater than about 1.0; (iii) a Class III drug, optionally characterized by an Papp of less than about 2×10⁻⁶ (cm/sec) and a Q of less than or equal to about 0.5; and (iv) a Class IV drug, optionally characterized by an Papp of less than about 2×10⁻⁶ (cm/sec) and a Q of greater than about 1.0. In some embodiments, the active agent is selected from the group consisting of a vitamin, a curcuminoid, a cannabinoid, a plant alkaloid, quinone, derivatives thereof, salts thereof, and combinations thereof.

In some embodiments, the composition comprises: at least one cannabinoid, optionally wherein the at least one cannabinoid is selected from the group consisting of cannabidiol (CBD), cannabidiolic acid (CBDA), cannabinol (CBN), cannabinolic acid (CBNA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabivarinic acid (CBVA), tetrahydrocannabivarin (THCV), tetrahydrocannabivarinic acid (THCVA) cannabidivarin (CBDV), cannabidivarinic acid (CBDVA), cannabichromevarin (CBCV), cannabichromevarinic acid (CBCVA), cannabigerovarin (CBGV), cannabigerovarinic acid (CBGVA), salt thereof, derivatives thereof, and combinations thereof; at least one curcuminoid, optionally wherein the at least one curcuminoid is selected from the group consisting of curcumin, demethoxycurcumin (DMC), bis-demethoxycurcumin (BDMC), salts thereof, derivatives thereof, and combinations thereof; at least one vitamin, optionally wherein the at least one vitamin is selected from the group consisting of vitamin A, vitamin C, vitamin D2 (ergocalciferol), vitamin D3 (cholecalciferol), vitamin E (α-tocopherol), vitamin K, vitamin K1, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folate), vitamin B12 (cyanocobalamin), salts thereof, derivatives thereof, and combinations thereof; at least one plant alkaloid, optionally wherein the plant alkaloid is selected from the group consisting of berberine, salts thereof, derivatives thereof, and combinations thereof; and/or at least one quinone, optionally wherein the quinone is selected from the group consisting of Coenzyme Q10 (CoQ10), salts thereof, derivatives thereof, and combinations thereof.

In some embodiments, the active agent is present in an amount of about 0.01% w/v to about 50% w/v, optionally wherein the composition comprises: an active agent in an amount of about 0.01% w/v to about 15% w/v; an active agent in an amount of about 0.01% w/v to about 10% w/v; an active agent in an amount of about 0.01% w/v to about 5% w/v; an active agent in an amount of about 0.01% w/v to about 1% w/v; an active agent in an amount of about 0.1% w/v to about 1% w/v; an active agent in an amount of about 0.5% w/v to about 5% w/v; an active agent in an amount of about 5% w/v to about 15% w/v; an active agent in an amount of about 10% w/v to about 15% w/v; an active agent in an amount of about 1% w/v to about 3% w/v; an active agent in an amount of about 2% w/v; or an active agent in an amount of about 1% w/v. In some embodiments, the lithe liposome composition comprises a plurality of active agents (e.g., more than one active agent, e.g., at least two active agents). In some embodiments, each active agent in a plurality of active agents is independently present in an amount of about 0.01% w/v to about 50% w/v. In such embodiments, the amount of active agent in a plurality of active agents may be greater than about 50% w/v.

In some embodiments, the composition comprises a sterol or a derivative thereof, optionally wherein the sterol or the derivative thereof comprises a cholesterol or a derivative thereof. In some embodiments, the sterol or the derivative thereof is present in an amount of about 0.01% w/v to about 50% w/v, optionally wherein the composition comprises: a sterol or a derivative thereof in an amount of about 0.01% w/v to about 15% w/v; a sterol or a derivative thereof in an amount of about 0.01% w/v to about 10% w/v; a sterol or a derivative thereof in an amount of about 0.01% w/v to about 5% w/v; a sterol or a derivative thereof in an amount of about 0.01% w/v to about 1% w/v; a sterol or a derivative thereof in an amount of about 0.1% w/v to about 1% w/v; a sterol or a derivative thereof in an amount of about 0.5% w/v to about 5% w/v; a sterol or a derivative thereof in an amount of about 5% w/v to about 15% w/v; a sterol or a derivative thereof in an amount of about 10% w/v to about 15% w/v; a sterol or a derivative thereof in an amount of about 1% w/v to about 3% w/v; a sterol or a derivative thereof in an amount of about 2% w/v; or a sterol or a derivative thereof in an amount of about 1% w/v. In some embodiments, the composition is substantially free of a cholesterol or a derivative thereof.

In some embodiments, the composition comprises (i) a phospholipid, an active agent, a cholesterol or a derivative thereof, a lipid bilayer modifier and/or a hydrophilic matrix; (ii) a phospholipid, an active agent, a cholesterol or a derivative thereof, a lipid bilayer modifier, an absorption enhancer, and/or a hydrophilic matrix; (iii) a phospholipid, an active agent, a cholesterol or a derivative thereof, a lipid bilayer modifier, optionally comprising a surfactant, an absorption enhancer, an additional lipid bilayer modifier, optionally comprising a solvent, and/or a hydrophilic matrix. (iv) a phospholipid, an active agent, a lipid bilayer modifier, and/or a hydrophilic matrix; or (v) a phospholipid, an active agent, a lipid bilayer modifier, an absorption enhancer, and/or a hydrophilic matrix.

In some embodiments, the composition comprises a phospholipid comprising a phosphatidylcholine (PC) or a derivative thereof; an active agent comprising a cannabidiol (CBD) or a derivative thereof; a cholesterol or a derivative thereof; a lipid bilayer modifier comprising span 60 or a derivative thereof; and/or a hydrophilic matrix.

In some embodiments, the composition comprises a phospholipid comprising a phosphatidylcholine (PC) or a derivative thereof; an active agent comprising a cannabidiol (CBD) or a derivative thereof; a cholesterol or a derivative thereof; a lipid bilayer modifier comprising a polysorbate 80 or a derivative thereof; an absorption enhancer comprising a quercetin or a derivative thereof; and/or a hydrophilic matrix.

In some embodiments, the composition comprises a phospholipid comprising a phosphatidylcholine (PC) or a derivative thereof; an active agent comprising a berberine hydrochloride or a derivative thereof; a lipid bilayer modifier comprising a bile acid, a bile salt, or a derivative thereof; and/or a hydrophilic matrix.

In some embodiments, the composition comprises a phospholipid comprising a phosphatidylcholine (PC) or a derivative thereof; an active agent comprising a Vitamin D3 or a derivative thereof; a cholesterol or a derivative thereof; a lipid bilayer modifier comprising a sodium cholate or a derivative thereof; and/or a hydrophilic matrix.

In some embodiments, the composition comprises a phospholipid comprising a phosphatidylcholine (PC) or a derivative thereof; an active agent comprising a Vitamin D3 or a derivative thereof; a cholesterol or a derivative thereof; an absorption enhancer comprising a dihydroquercetin or a derivative thereof; a lipid bilayer modifier comprising sodium cholate; and/or a hydrophilic matrix.

In some embodiments, the composition comprises a phospholipid comprising a phosphatidylcholine (PC) or a derivative thereof; an active agent comprising a Vitamin D3 or a derivative thereof; a cholesterol or a derivative thereof; an absorption enhancer comprising menthol; a lipid bilayer modifier comprising sodium cholate or a derivative thereof; and/or a hydrophilic matrix. 31. The composition of any one of the preceding claims, comprising: a phospholipid comprising a phosphatidylcholine (PC) or a derivative thereof; an active agent comprising a Vitamin D3 or a derivative thereof; a cholesterol or a derivative thereof; a lipid bilayer modifier comprising a sodium cholate or a derivative thereof; and/or a hydrophilic matrix.

In some embodiments, the composition comprises a phospholipid comprising a phosphatidylcholine (PC) or a derivative thereof; an active agent comprising a cannabidiol (CBD) or a derivative thereof; a cholesterol or a derivative thereof; a lipid bilayer modifier comprising a surfactant, optionally wherein the surfactant comprises span 80 or a derivative thereof; an absorption enhancer comprising a dihydroquercetin or a derivative thereof; a lipid bilayer modifier comprising a solvent, optionally wherein the solvent comprises methanol or a derivative thereof; and/or a hydrophilic matrix.

In some embodiments, the composition comprises a phospholipid comprising a phosphatidylcholine (PC) or a derivative thereof; an active agent comprising a Vitamin K1 or a derivative thereof; a lipid bilayer modifier comprising a bile acid, a bile salt, or a derivative thereof; an absorption enhancer comprising a quercetin or a derivative thereof; and/or a hydrophilic matrix.

In some embodiments, the composition comprises a phospholipid comprising a phosphatidylcholine (PC) or a derivative thereof; an active agent comprising a curcuminoid or a derivative thereof; a lipid bilayer modifier comprising a hydroxypropyl-β-cyclodextrin (HBC) or a derivative thereof; and/or a hydrophilic matrix.

In some embodiments, the composition comprises a phospholipid comprising a phosphatidylcholine (PC) or a derivative thereof; an active agent comprising a curcuminoid or a derivative thereof; a lipid bilayer modifier comprising a bile acid, a bile salt, or a derivative thereof; an absorption enhancer comprising a quercetin or a derivative thereof; and/or a hydrophilic matrix.

In some embodiments, the lithe liposome composition comprises a phospholipid, optionally a phosphatidylcholine (PC) or a derivative thereof, in an amount of about 1.9% w/v; an active agent, optionally a cannabidiol (CBD) or a derivative thereof, in an amount of about 0.2% w/v; a cholesterol or a derivative thereof in an amount of about 0.1% w/v; a lipid bilayer modifier, optionally span 60 or a derivative thereof, in an amount of about 0.5% w/v; and/or quantum satis (QS) a hydrophilic matrix.

In some embodiments, the lithe liposome composition comprises a phospholipid, optionally a phosphatidylcholine (PC) or a derivative thereof, in an amount of about 2.0% w/v; an active agent, optionally a cannabidiol (CBD) or a derivative thereof, in an amount of about 0.2% w/v; a cholesterol or a derivative thereof in an amount of about 0.1% w/v; a lipid bilayer modifier, optionally a polysorbate 80 or a derivative thereof, in an amount of about 0.5% w/v; an absorption enhancer, optionally a quercetin or a derivative thereof, in an amount of about 0.2% w/v; and/or quantum satis (QS) a hydrophilic matrixquantum satis (QS) a hydrophilic matrix.

In some embodiments, the lithe liposome composition comprises a phospholipid, optionally a phosphatidylcholine (PC) or a derivative thereof, in an amount of about 2.1% w/v; an active agent, optionally a berberine hydrochloride or a derivative thereof, in an amount of about 0.1% w/v; a lipid bilayer modifier, optionally a bile acid, a bile salt, or a derivative thereof, in an amount of about 0.3% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.16% w/v; an active agent, optionally a Vitamin D3 or a derivative thereof, in an amount of about 0.08% w/v; a cholesterol or a derivative thereof in an amount of about 0.02% w/v; a lipid bilayer modifier, optionally a sodium cholate or a derivative thereof, in an amount of about 0.04% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.16% w/v; an active agent, optionally a Vitamin D3, in an amount of about 0.08% w/v; a cholesterol or a derivative thereof in an amount of about 0.02% w/v; an absorption enhancer, optionally a dihydroquercetin or a derivative thereof, in an amount of about 0.05% w/v; a lipid bilayer modifier, optionally sodium cholate, in an amount of about 0.04% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.16% w/v; an active agent, optionally a Vitamin D3, in an amount of about 0.08% w/v; a cholesterol or a derivative thereof in an amount of about 0.02% w/v; an absorption enhancer, optionally menthol, in an amount of about 0.02% w/v; a lipid bilayer modifier, optionally sodium cholate or a derivative thereof, in an amount of about 0.04% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.06% w/v; an active agent, optionally a Vitamin D3 or a derivative thereof, in an amount of about 0.03% w/v; a cholesterol or a derivative thereof in an amount of about 0.01% w/v; a lipid bilayer modifier, optionally a sodium cholate or a derivative thereof, in an amount of about 0.01% w/v; and/or quantum satis (QS) a hydrophilic matrix.

In some embodiments, the lithe liposome composition comprises a phospholipid, optionally a phosphatidylcholine (PC) or a derivative thereof, in an amount of about 1.9% w/v; an active agent, optionally a cannabidiol (CBD) or a derivative thereof, in an amount of about 0.2% w/v; a cholesterol or a derivative thereof in an amount of about 0.2% w/v; a lipid bilayer modifier comprising a surfactant, optionally span 80 or a derivative thereof, in an amount of about 0.7% w/v; an absorption enhancer, optionally a dihydroquercetin or a derivative thereof, in an amount of about 0.2% w/v; a lipid bilayer modifier comprising a solvent, optionally methanol or a derivative thereof, in an amount of about 0.2% w/v; and/or quantum satis (QS) a hydrophilic matrix.

In some embodiments, the lithe liposome composition comprises a phospholipid, optionally a phosphatidylcholine (PC) or a derivative thereof, in an amount of about 3.0% w/v; an active agent, optionally a Vitamin K1 or a derivative thereof, in an amount of about 2.25% w/v; a lipid bilayer modifier, optionally a bile acid, a bile salt, or a derivative thereof, in an amount of about 1.5% w/v; an absorption enhancer, optionally a quercetin or a derivative thereof, in an amount of about 0.125% w/v; and/or quantum satis (QS) a hydrophilic matrix.

In some embodiments, the lithe liposome composition comprises a phospholipid, optionally a phosphatidylcholine (PC) or a derivative thereof, in an amount of about 2.0% w/v; an active agent, optionally a curcuminoid or a derivative thereof, in an amount of about 0.25% w/v; a lipid bilayer modifier, optionally a hydroxypropyl-β-cyclodextrin (HBC) or a derivative thereof, in an amount of about 1.0% w/v; and/or quantum satis (QS) a hydrophilic matrix.

In some embodiments, the lithe liposome composition comprises a phospholipid, optionally a phosphatidylcholine (PC) or a derivative thereof, in an amount of about 12.5% w/v; an active agent, optionally a curcuminoid or a derivative thereof, in an amount of about 2% w/v; a lipid bilayer modifier, optionally a bile acid, a bile salt, or a derivative thereof, in an amount of about 10% w/v; an absorption enhancer, optionally a quercetin or a derivative thereof, in an amount of about 0.5% w/v; and/or quantum satis (QS) a hydrophilic matrix.

In some embodiments, the composition is an oral composition.

In some embodiments, the composition is characterized by improved solubility of an active agent as compared to a reference composition, optionally wherein the reference composition comprises an oil-based composition, a micellized composition, and/or a conventional liposome, optionally wherein the solubility is tested using a buffer selected from the group consisting of phosphate-buffered saline (PBS), simulated gastric fluid (SGF), fasted state simulated intestinal fluid (FaSSIF), and fed state simulated intestinal fluid (FeSSIF).

In some embodiments, the composition is characterized by improved passive, transcellular permeability and/or permeability across intestinal epithelia as compared to a reference composition, optionally wherein the reference composition comprises an oil-based composition, a micellized composition, and/or a conventional liposome, optionally wherein the permeability is tested using a buffer selected from the group consisting of phosphate-buffered saline (PBS), simulated gastric fluid (SGF), fasted state simulated intestinal fluid (FaSSIF), and fed state simulated intestinal fluid (FeSSIF).

In some embodiments, the composition is characterized by improved stability in the gastrointestinal tract as compared to a reference composition, optionally wherein the reference composition comprises an oil-based composition, a micellized composition, and/or a conventional liposome.

In some embodiments, the composition is characterized by improved oral absorption of the active agent as compared to a reference composition, optionally wherein the reference composition comprises an oil-based composition, a micellized composition, and/or a conventional liposome.

In some embodiments, the composition is characterized by improved oral bioavailability of the active agent as compared to a reference composition, optionally wherein the reference composition comprises an oil-based composition, a micellized composition, and/or a conventional liposome.

In some embodiments, the composition is characterized by improved bio-variability as compared to a reference composition, optionally wherein the reference composition comprises an oil-based composition, a micellized composition, and/or a conventional liposome.

In one aspect, the disclosure features a pharmaceutical composition comprising the lithe liposome composition described herein and optionally a pharmaceutically acceptable carrier. In some embodiments, the pharmaceutical composition is an oral pharmaceutical composition.

In one aspect, the disclosure features a method of preparing a lithe liposome composition, comprising: (i) dissolving a phospholipid in an organic solvent to form a lipid mixture; (ii) optionally contacting the lipid mixture with a lipid bilayer modifier, an absorption enhancer, or a combination thereof; (iii) optionally contacting the lipid mixture with an active agent, e.g., one or more active agent(s); (iv) drying the lipid mixture to remove the organic solvent to form a dried lipid thin film; (v) hydrating the dried lipid thin film with a hydrophilic solution (e.g., an aqueous solution) to form a liposomal suspension; (vi) optionally contacting the liposomal suspension with a lipid bilayer modifier, an absorption enhancer, or a combination thereof; (vii) optionally contacting the liposomal suspension with one or more hydrophilic active agent(s); and (viii) optionally agitating, stirring, and/or sonicating the liposomal suspension.

In one aspect, the disclosure features a method of enhancing the stability of an active agent in the gastrointestinal tract of a subject in need thereof, comprising administering the lithe liposome composition described herein, or the pharmaceutical composition described herein to the subject. In some embodiments, the administration comprises oral administration.

In one aspect, the disclosure features a method of improving the permeability of an active agent across the intestinal epithelia of a subject in need thereof, comprising administering the lithe liposome composition described herein, or the pharmaceutical composition described herein to the subject. In some embodiments, the administration comprises oral administration.

In one aspect, the disclosure features a method of improving the oral absorption, the oral bioavailability, and/or the bio-variability of an active agent in a subject in need thereof, comprising administering the lithe liposome composition described herein, or the pharmaceutical composition described herein to the subject. In some embodiments, the administration comprises oral administration. In one aspect, the disclosure features a method of treating and/or preventing a disease or condition in a subject in need thereof, comprising administering the lithe liposome composition described herein, or the pharmaceutical composition described herein to the subject. In some embodiments, the administration comprises oral administration.

The present invention is further illustrated by the following detailed description and drawings.

### BRIEF DESCRIPTION OF THE DRAWINGS

FIG. 1 is a graph that depicts the comparative in vitro release of the active agent, Vitamin D3, from an oil-based reference composition (Reference 1); an oil-based reference composition comprising sunflower lecithin (Reference 2); and a lithe liposome composition comprising a lipid bilayer modifier (e.g., sodium cholate) (Example 4). These data demonstrate that lithe liposome compositions comprising a lipid bilayer modifier (Example 4) in the absence of an absorption enhancer can enhance the in vitro release of an active agent.

FIG. 2 is a graph that depicts the comparative in vitro release of the active agent, Vitamin D3, from a lithe liposome composition comprising a lipid bilayer modifier (e.g., sodium cholate) (Example 4); a lithe liposome composition comprising a lipid bilayer modifier (e.g., sodium cholate) and an absorption enhancer (e.g., dihydroquercetin) (Example 5); and a lithe liposome composition comprising a lipid bilayer modifier (e.g., sodium cholate) and an absorption enhancer (e.g., menthol) (Example 6). These data demonstrate that lithe liposome compositions comprising both a lipid bilayer modifier (e.g., sodium cholate) and an absorption enhancer (e.g., a flavonoid, such as dihydroquercetin, or a terpene, such as menthol) can further enhance the in vitro release of an active agent as compared to lithe liposome compositions comprising only a lipid bilayer modifier (e.g., sodium cholate).

FIG. 3 is a transmission electron microscopy (TEM) image of a Vecell liposomal vesicle with characteristic lipid bilayer structure.

FIG. 4 is a series of photographs of the solubility test showing that Vecell Liposomal Curcuminoids is soluble in PBS buffer from 0 to 24 hours, while undissolved particles appear in On-Market Absorption-enhanced product throughout 24 hours. The solubility results are also summarized in Table 2 and demonstrate a significant increase of curcuminoids solubility in all four bio-relevant media tested.

FIG. 5 is a graph that depicts the permeability of Curcuminoids in Vecell formulation is greatly enhanced by more than 10 times in all 4 bio-relevant buffers. Unlike the reference formulation, the permeability of curcuminoids is more consistent in FaSSIF and FeSSIF, indicating a more comparable absorption under fast and fed conditions.

FIG. 6 is a series of graphs showing serum Vitamin D3 and 25-hydroxy Vitamin D3 levels following one single oral administration of VeCell Liposomal Vitamin D3, Classic Liposomal Vitamin D3 and On-Market Oil-Based Vitamin D3 formulations (n=6 per group) in rats. Each point presented is the mean and standard deviation for each group at times points 0, 2, 4, 8, 16, and 24 days.

FIG. 7 is a series of graphs showing Area Under Curve (AUC(0-24 h)) for serum Vitamin D3 and 25-hydroxy Vitamin D3 levels following one single dose of oral administration of VeCell Liposomal Vitamin D3, classic liposomal Vitamin D3 and On-Market Oil-Based Vitamin D3 formulations (n=6 per group) in rats. The bars show the mean and standard deviation. One-way ANOVA and post hoc Tukey tests were used for statistical changes ****p<0.0001 as compared to OBF group; ####p<0.0001 as compared to CLP group.

FIG. 8 is a series of graphs showing the concentration and Area Under Curve (AUC(0-24 hr)) for Serum Vitamin K levels following oral administration of VeCell Liposomal Vitamin K, Unencapsulated Vitamin K and On-Market Oil-Based Vitamin K formulations (n=6 per group) in rats. Each point presented is the mean and standard deviation for each group at 0, 1, 2, 4, 6, 8, 16, and 24 hours after a single dose. One-way ANOVA and post hoc Tukey tests were used for statistical changes ****p<0.0001 as compared to oil-based formulation group; ####p<0.0001 as compared to unencapsulated group.

FIG. 9 is a series of graphs showing the concentration and Area Under Curve (AUC(0-144 hr)) for serum Vitamin K levels following oral administration of VeCell Liposomal Vitamin K formulation and On-Market Oil-Based Vitamin K (n=6 per group) in rats. Vitamins were administrated at 0, 24, and 48 hours. The feeding was stopped after 48 hours. Each point presented is the mean and standard deviation for each group at 0, 1, 2, 4, 6, 8, 16, 24, 28, 48, 52, 72, 96, 120 and 144 hours. One-way ANOVA and post hoc Tukey tests were used for statistical changes ****p<0.0001 as compared to oil-based formulation group; ####p<0.0001 as compared to unencapsulated group.

### DETAILED DESCRIPTION

The present disclosure is based, at least in part, on the discovery of lithe liposome (also referred to as “Vecell”) compositions effective for the oral delivery of active agents. Various factors influence the oral bioavailability of an active agent including, for example, the stability, solubility, and permeability, of the active agent in the microenvironment of the gastrointestinal (GI) tract. Conventional drug delivery systems (e.g., an oil-based composition, a micellized composition, and/or a conventional liposome) can be associated with poor permeability of active agent, for example, across intestinal epithelia due to their limited ability to enhance active agent solubility and the presence of gastrointestinal mucus, which can trap active agent and reduce the permeability and absorption of orally administered active agent. The lithe liposome compositions described herein can improve the oral bioavailability of an active agent as compared to delivery of the active agent in the absence of the lithe liposome composition, for example, by using conventional drug delivery systems (e.g., an oil-based composition, a micellized composition, and/or a conventional liposome). In certain embodiments, the lithe liposome compositions described herein can improve the oral bioavailability of an active agent by modulating, e.g., increasing, the stability, solubility, and/or permeability of the active agent for effective oral delivery.

The present invention is based, at least in part, on the discovery of a lithe liposome composition including lipid bilayer modifiers and/or absorption enhancers so as to overcome the challenges associated with oral delivery using conventional drug delivery systems (e.g., an oil-based composition, a micellized composition, and/or a conventional liposome).

Accordingly, the present disclosure provides compositions (e.g., pharmaceutical and nutraceutical compositions), comprising a lithe liposome. Such lithe liposome compositions are effective for the oral delivery of active agents. In some embodiments, the lithe liposome compositions can comprise an absorption enhancer to modulate, e.g., increase, the stability, solubility, permeability, and/or absorption of an active agent for effective oral delivery. In some embodiments, the lithe liposome compositions can comprise a lipid bilayer modifier to modulate, e.g., increase, the elasticity of the liposome by changing the fluidity, permeability, and/or stability of the lipid bilayer. The present disclosure also provides systems (e.g., drug delivery systems), comprising the lithe liposome compositions, and methods of making and using the lithe liposome compositions.

The lithe liposome compositions of the present disclosure provide, inter alia, a controllable means to fine-tune the oral bioavailability of an active agent, e.g., by modulating, e.g., increasing, the stability, solubility, permeability, and/or absorption of encapsulated active agent. The lithe liposome compositions are designed to achieve effective oral delivery of active agent. Delivery of an active agent using the lithe liposome compositions described herein can improve the oral bioavailability of the active agent as compared to delivery of the active agent in the absence of the lithe liposome composition, for example, by using a conventional drug delivery system, such as an oil-based composition and/or a micellized composition. Such lithe liposome compositions can overcome the challenges associated with oral delivery using conventional drug delivery systems, such as active agent instability in the gastrointestinal tract and poor intestinal permeability and/or absorption of active agent. Thus, the use of the lithe liposome compositions can improve the oral absorption, oral bioavailability, and/or bio-variability of active agents, e.g., as compared to the use of a reference composition, such as a conventional drug delivery system (e.g., an oil-based composition, a micellized composition, and/or a conventional liposome). Various aspects of the present disclosure are described in further detail in the following subsections:

### Definitions

Unless defined otherwise, all technical and scientific terms used herein have the same meaning as commonly understood by one of ordinary skill in the art to which the invention pertains.

As used in this specification and the appended claims, the singular forms “a,” “an,” and “the” include plural references unless the content clearly dictates otherwise.

The articles “a” and “an” are used herein to refer to one or to more than one (e.g., to at least one) of the grammatical object of the article. By way of example, “an element” means one element or more than one element, e.g., a plurality of elements.

The use of the alternative (e.g., “or”) should be understood to mean either one, both, or any combination thereof of the alternatives. The term “or” is used herein to mean, and is used interchangeably with, the term “and/or,” unless context clearly indicates otherwise.

As used herein, the term “about,” when referring to a measurable value such as an amount, a temporal duration, and the like, is meant to encompass variations of ±20%, or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” “comprises,” and “comprised of” are meant to be synonymous with “include,” “including,” “includes,” or “contain,” “containing,” “contains” and are inclusive or open-ended terms that specifies the presence of what follows, e.g., component and do not exclude or preclude the presence of additional, non-recited components, features, element, members, steps, known in the art or disclosed therein. By way of example, the term “including” is used herein to mean, and is used interchangeably with, the phrase “including but not limited to.”

As used herein, the terms “such as,” “for example,” and the like are intended to refer to exemplary embodiments and not to limit the scope of the present disclosure.

Recitation of ranges of values herein are merely intended to serve as a shorthand method of referring individually to each separate value recited or falling within the range, unless otherwise indicated herein, and each separate value is incorporated into the specification as if it were individually recited.

The term “at least” prior to a number or series of numbers is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range. As used herein, ranges include both the upper and lower limit.

As used herein, the term “derivative” refers to a compound that may be derived from another compound, e.g., by a chemical reaction. In some embodiments, a “derivative” refers to a compound that has been produce from another compound by replacing one or more atoms or groups of atoms with different atoms or groups of atoms. In some embodiments, a “derivative” may result from functionalization, substitution, redox manipulation, unsaturation and/or ring incorporation of the original compound. In some embodiments, a “derivative” refers to a structural analogue having a structure similar to that of the original compound, but differing from it in respect to at least one component (e.g., differing by one or more atoms, functional groups, or substructures). In some embodiments, a “derivative” refers to a functional analogue having similar physical, chemical, biochemical, and/or pharmacological properties to that of the original compound. In some embodiments, a structural analog may not necessarily be a functional analog, and may have different physical, chemical, biochemical, or pharmacological properties to that of the original compound. In some embodiments, “derivative” refers to a metabolite. In some embodiments, the term “derivative” is intended to encompass salt forms, hydrates, pro-drugs, solvates, racemic mixtures, specific stereoisomers, crystalline, and amorphous forms of a compound.

As used herein, the various forms of the term “modulate” or “alter” are intended to include stimulation, activation, and/or enhancement (e.g., increasing or upregulating a particular response, level, or activity) and inhibition (e.g., decreasing or downregulating a particular response, level, or activity).

In certain embodiments, the lithe liposome compositions described herein can improve the oral bioavailability of an active agent by modulating, e.g., increasing, the stability, solubility, and/or permeability of the active agent by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% or more, e.g., as compared to the use of a reference composition, such as a conventional drug delivery system (e.g., an oil-based composition, a micellized composition, and/or a conventional liposome).

In certain embodiments, the lithe liposome compositions described herein can improve the oral bioavailability of an active agent by modulating, e.g., increasing, the stability, solubility, and/or permeability of the active agent by at least about 2-fold, or at least about 3-fold, or at least about 4-fold, or at least about 5-fold, or at least about 10-fold or more, e.g., as compared to the use of a reference composition, such as a conventional drug delivery system (e.g., an oil-based composition, a micellized composition, and/or a conventional liposome).

As used herein, the term “drug delivery system” refers to a composition for administering an active agent to a subject in need thereof, such as a human or an animal. In some embodiments, the drug delivery system comprises a lithe liposome of the present disclosure. In some embodiments, the drug delivery system comprising a lithe liposome of the present disclosure which releases an active agent into a cell, e.g., a target cell, of a subject during drug delivery to enhance the bioavailability, efficacy, safety, and/or pharmacokinetics of the active agent.

As used herein, the term “conventional drug delivery system” refers to a drug delivery system that does not include a lithe liposome of the present disclosure. In some embodiments, the conventional drug delivery system comprises an oil-based composition, a micellized composition, and/or a conventional liposome.

The terms “decrease,” “reduced,” “reduction,” or “inhibit” are all used herein to mean a decrease by a statistically significant amount. In some embodiments, the terms “reduced,” “reduction,” “decrease,” or “inhibit” can mean a decrease by at about least 5% as compared to a reference level, for example a decrease by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% or more as compared to a reference level. In some embodiments, the terms can represent a 100% decrease, e.g., a non-detectable level as compared to a reference level.

The terms “increased,” “increase,” “enhance,” or “activate” are all used herein to mean an increase by a statically significant amount. In some embodiments, the terms “increased,” “increase,” “enhance,” or “activate” can mean an increase of at least about 5% as compared to a reference level, for example an increase of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 250%, at least about 500%, or at least about 1000% or more. In some embodiments, the terms can represent an increase up to and including a 100% increase as compared to a reference level, or at least about a 2-fold, or at least about a 3-fold, or at least about a 4-fold, or at least about a 5-fold, or at least about a 10-fold increase, or at least about a 100-fold increase, or at least about a 1000-fold increase, or any increase between 2-fold and 1000-fold or greater as compared to a reference level. In the context of a marker or symptom, an “increase” is a statistically significant increase in such level.

As used herein, “substantially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein, the terms “preventing” or “prevention” refers to a reduction in risk of acquiring a condition, disease, and/or disorder (e.g., causing at least one of the clinical symptoms of the condition, disease, and/or disorder not to develop in a patient that may be exposed to or predisposed to the condition, disease, and/or disorder but does not yet experience or display symptoms of the condition, disease, and/or disorder).

As used herein, the terms “treating” or “treatment” refer to a beneficial or desired result including, but not limited to, alleviation or amelioration of one or more symptoms of a condition, disease, and/or disorder, diminishing the extent of a condition, disease, and/or disorder, stabilized (e.g., not worsening) state of a condition, disease, and/or disorder, amelioration or palliation of a condition, disease, and/or disorder, whether detectable or undetectable. “Treatment” can also mean prolonging survival as compared to expected survival in the absence of treatment.

As used herein, the term “prophylactically effective amount,” is intended to include the amount of an active agent that, when administered to a subject who does not yet experience or display symptoms of a condition, disease, and/or disorder, but who may be predisposed to the condition, disease, and/or disorder, is sufficient to prevent or ameliorate the condition, disease, and/or disorder or one or more symptoms of the condition, disease, and/or disorder. Ameliorating the condition, disease, and/or disorder includes slowing the course of the condition, disease, and/or disorder or reducing the severity of later-developing condition, disease, and/or disorder. The “prophylactically effective amount” may vary depending on the active agent, how the active agent is administered, the degree of risk of the condition, disease, and/or disorder, and the history, age, weight, family history, genetic makeup, the types of preceding or concomitant treatments, if any, and other individual characteristics of the patient to be treated.

A “therapeutically effective amount” or “prophylactically effective amount” also includes an amount of an active agent that produces some desired local or systemic effect at a reasonable benefit/risk ratio applicable to any treatment. Active agents employed in the compositions and methods of the present disclosure may be administered in a sufficient amount to produce a reasonable benefit/risk ratio applicable to such treatment.

As used herein, the term “administering” to a subject includes dispensing, delivering or applying a composition as described herein to a subject by any suitable route for delivery of the composition to the subject. Exemplary modes of administration include, but are not limited to, injection, infusion, instillation, inhalation, or ingestion. Alternatively or in combination, delivery is by the oral, parenteral route, intracerebral injection, intramuscular injection, subcutaneous injection, intradermal injection, intravenous injection, buccal administration, and/or administration by the rectal, colonic, vaginal, intranasal, or respiratory tract route.

As used herein, the terms “bio-variability” or “biological variation” or “biological variance” refers to the appearance of differences in the magnitude of response among subjects in the same population given the same dose of an active agent.

The term “biopharmaceutical classification system” or “BCS” as used herein, refers to a scientific classification framework for drug substances based on their aqueous solubility and intestinal permeability (US Dept. Health & Human Services, Food and Drug Administration Center for Drug Evaluation and Research (CDER) August 2000). In some embodiments, the active agent comprises a class of the biopharmaceutical classification system (BCS). In some embodiments, a BCS class is selected from the group consisting of Class I (High permeability, High solubility); Class II (Low solubility, Low Permeability); Class III (High Solubility, Low Permeability); or Class IV (Low solubility, Low permeability). According to BCS system, drugs are classified into four types based on their intestinal permeability and solubility. BCS classification is based, for example, on parameters including solubility, dissolution rate and permeability, which control absorption. In case of class I drugs, absorption is maximum; class II drugs are solubility limited; class III drugs are permeability limited; and class IV drugs have poorly absorbed, as described in Table 1. Apparent permeability index (Papp) is the index used to assess the degree of permeability of drug substances. The permeability coefficient, which is a measure of flow to the drug concentration in the donor compartment. Papp of any drug substance can be calculated using in vitro, ex vivo, in situ, and in vivo techniques. See, e.g., Samineni et al. (Emerging Role of Biopharmaceutical Classification and Biopharmaceutical Drug Disposition System in Dosage form Development: A Systematic Review. Turk J Pharm Sci. 2022 Dec. 21; 19(6):706-713), the contents of which are incorporated herein by reference in their entirety.

TABLE 1
BCS classification system

| Class | Solubility | Permeability | *Papp (cm/sec) | *Q | Significance |
|---|---|---|---|---|---|
| I | High | High | Papp > 10⁻⁵ | q ≤ 0.5 | Well absorbed |
| II | Low | High | Papp > 10⁻⁵ | q > 1 | Solubility |
| | | | | | limited |
| | High | Low | Papp < 2 × 10⁻⁶ | q ≤ 0.5 | Permeability |
| | | | | | limited |
| IV | Low | Low | Papp < 2 × 10⁻⁶ | q > 1 | Poorly |
| | | | | | absorbed |

*Pₐₚₚ: Apparent permeability, *Q: Dose/solubility, BCS: Biopharmaceutical classification system

The term “permeability” as used herein, refers to any material that permits liquids or gases to pass through. In some embodiments, the term “permeability” is intended to encompass “intestinal permeability.”

As used here, the term “intestinal permeability” refers to the passage of materials from the gastrointestinal tract, through the intestinal epithelium, to the systemic circulation and thereby the rest of a subject's body. The term “intestinal permeability” encompasses the permeability of the intestinal mucosa of a subject, permitting the absorption of vital nutrients and, e.g., active agents, from the gut lumen while presenting a barrier against the passage of pathogenic substances into the subject's body. Passage through the intestinal epithelium typically occurs via a transepithelial/transcellular route (e.g., through epithelial cells) or a paracellular route (e.g., between epithelial cells). Paracellular passage is the predominant route of intestinal epithelium passage of water and solutes, with tight junctions being the most important regulators of paracellular passage.

The term “solubility” as used herein, refers to the amount of a substance that will dissolve in a given amount of another substance. Typically, solubility is expressed as the number of parts by weight dissolved by 100 parts of solvent at a specified temperature and pressure or as percent by weight or by volume.

In some embodiments, the term “batch,” as used herein, may refer to an amount (e.g., therapeutically effective amount and/or a prophylactically effective amount) of a composition described herein. In some embodiments, the term “batch,” as used herein, may refer to a group of dosage forms (e.g., oral dosage forms) comprising an amount (e.g., therapeutically effective amount and/or a prophylactically effective amount) of a composition described herein. In some embodiments, the term “batch,” as used herein, may refer to a commercial batch of a composition described herein.

As used herein, the term “commercial batch” or “manufacturing batch” may refer to a relatively large quantity of a composition described herein that is produced to meet demand for commercial products comprising (e.g., specific dosage forms, such as oral dosage forms) comprising an amount (e.g., therapeutically effective amount and/or a prophylactically effective amount) of the composition.

In some embodiments, a “commercial batch” may refer to a batch having a volume of about 5 mL or greater. In some embodiments, a “commercial batch” may refer to a batch having a volume of about 5 mL to about 5000 L. In one embodiments, a “commercial batch” may refer to a batch having a volume of about 5 mL to about 1000 mL (e.g., about 5 mL, about 25 mL, about 50 mL, about 75 mL, about 100 mL, about 125 mL, about 150 mL, about 175 mL, about 200 mL, about 225 mL, about 250 mL, about 275 mL, about 300 mL, about 325 mL, about 350 mL, about 375 mL, about 400 mL, about 425 mL, about 450 mL, about 475 mL, about 500 mL, about 525 mL, about 550 mL, about 575 mL, about 600 mL, about 625 mL, about 650 mL, about 675 mL, about 700 mL, about 725 mL, about 750 mL, about 775 mL, about 800 mL, about 825 mL, about 850 mL, about 875 mL, about 900 mL, about 925 mL, about 950 mL, about 975 mL, or about 1000 mL). In one embodiments, a “commercial batch” may refer to a batch having a volume of about 1 L to about 5000 L (e.g., about 1 L, about 100 L, about 200 L, about 300 L, about 400 L, about 500 L, about 600 L, about 700 L, about 800 L, about 900 L, about 1000 L, about 1100 L, about 1200 L, about 1300 L, about 1400 L, about 1500 L, about 1600 L, about 1700 L, about 1800 L, about 1900 L, about 2000 L, about 2100 L, about 2200 L, about 2300 L, about 2400 L, about 2500 L, about 2600 L, about 2700 L, about 2800 L, about 2900 L, about 3000 L, about 3100 L, about 3200 L, about 3300 L, about 3400 L, about 3500 L, about 3600 L, about 3700 L, about 3800 L, about 3900 L, about 4000 L, about 4100 L, about 4200 L, about 4300 L, about 4400 L, about 4500 L, about 4600 L, about 4700 L, about 4800 L, about 4900 L, or about 5000 L). In some embodiments, a “commercial batch” may refer to a batch having a volume of greater than about 5000 L.

In some embodiments, a “commercial batch” may refer to a batch having a weight of about 1 kg or greater. In some embodiments, a “commercial batch” may refer to a batch having a weight of about 1 kg to about 1000 kg (e.g., about 1 kg, about 25 kg, about 50 kg, about 75 kg, about 100 kg, about 125 kg, about 150 kg, about 175 kg, about 200 kg, about 225 kg, about 250 kg, about 275 kg, about 300 kg, about 325 kg, about 350 kg, about 375 kg, about 400 kg, about 425 kg, about 450 kg, about 475 kg, about 500 kg, about 525 kg, about 550 kg, about 575 kg, about 600 kg, about 625 kg, about 650 kg, about 675 kg, about 700 kg, about 725 kg, about 750 kg, about 775 kg, about 800 kg, about 825 kg, about 850 kg, about 875 kg, about 900 kg, about 925 kg, about 950 kg, about 975 kg, or about 1000 kg). In some embodiments, a “commercial batch” may refer to a batch having a weight of greater than about 1000 kg.

In some embodiments, a “commercial batch” may refer to a batch having a volume and/or a weight sufficient for producing a predetermined number of dosage forms (e.g., oral dosage forms) comprising an amount (e.g., therapeutically effective amount and/or a prophylactically effective amount) of a composition described herein. In some embodiments, the predetermined number of dosage forms (e.g., oral dosage forms) may be at least about 1000. In some embodiments, the predetermined number of dosage forms (e.g., oral dosage forms) may be at least about 10000. In some embodiments, the predetermined number of dosage forms (e.g., oral dosage forms) may be at least about 100000. In some embodiments, the predetermined number of dosage forms (e.g., oral dosage forms) may be at about 1000 to about 100000.

In some embodiments, the term “batch size” may refer to the amount (e.g., by volume and/or weight) of a composition described herein present within a batch. In some embodiments, about 0.5% to about 100% (e.g., about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%) of a batch may be used to produce one or more dosage form(s).

As used herein, the term “batch manufacturing” generally refers to a production method that uses a step-by-step process for producing a specific quantity (e.g., a batch size) of a product, such as a composition, e.g., a pharmaceutical composition and/or a nutraceutical composition described herein. In some embodiments, the compositions described herein may be produced using batch manufacturing.

As used herein, the term “continuous manufacturing” generally refers to a production method that involves a single, uninterrupted process to create a product, such as a composition, e.g., a pharmaceutical composition and/or a nutraceutical composition described herein. In some embodiments, the compositions described herein may be produced using continuous manufacturing.

As used herein, the term “unit dosage form” or “dosage form” refers to physically discrete units suitable as unitary dosages for subjects, e.g., human subjects, each unit containing a predetermined quantity of an active agent and/or a composition comprising an active agent (e.g., a composition described herein), calculated in an amount sufficient to produce the desired biological effect in association with a pharmaceutically acceptable diluent, carrier, or vehicle. Examples of dosage forms include tablets, capsules, powders, oral solutions, and injectable solutions. An active agent may be available in multiple dosage forms.

As used herein, the term “oral dosage form” refers to a unit dosage form that may be orally administered. In some embodiments, the compositions described herein may be manufactured and/or administered as oral dosage forms. In some embodiments, the oral dosage forms may comprise an amount (e.g., a therapeutically effective amount and/or a prophylactically effective amount) of a composition described herein. In some embodiments, the oral dosage forms may further comprise one or more selected from the group consisting of a sweetener, such as sucralose, stevia extract, aspartame, and the like; a preservative, such as potassium sorbate, sodium benzoate, parabens, and the like; a natural flavor; an artificial flavor; a taste masking agent, such as citric acid, sodium bicarbonate, and the like; a viscosity builder, such as xanthan gum, starch, guar gum, hypromellose, also known as hydroxypropyl methylcellulose (HPMC), and the like; a colorant; and combinations thereof.

### Liposome Compositions

According to one aspect, the present disclosure provides liposome compositions, e.g., lithe liposome compositions.

### Liposomes

The compositions of the present disclosure comprise a liposome, e.g., a lithe liposome.

As used herein, the term “liposome” encompasses any compartment enclosed by a lipid bilayer, e.g., a phospholipid bilayer. Liposomes may also be referred to as lipid vesicles in the art. In general, liposomes may be characterized as vesicular systems comprising an aqueous internal environment bounded by one or more lipid bilayers, e.g., one or more phospholipid bilayers, formed when the lipids, e.g., the phospholipids, are dispersed into a hydrophilic matrix (e.g., an aqueous solution), such as water. Typically, the one or more lipid bilayers comprise lipids, e.g., phospholipids, containing an elongated non-polar portion (e.g., a hydrophobic tail) and a polar portion (e.g., hydrophilic head). The hydrophobic and hydrophilic portions of the molecule are preferably positioned at two ends of an elongated molecular structure. When lipids, e.g., phospholipids, are dispersed in a hydrophilic matrix (e.g., an aqueous solution), such as water, they can spontaneously form lipid bilayer membranes referred to as lamellae. The lamellae are composed of two mono layer sheets of lipid molecules with their non-polar (e.g., hydrophobic) surfaces facing each other and their polar (e.g., hydrophilic) surfaces facing the aqueous medium. The membranes formed by the lipids enclose a portion of the aqueous phase in a manner similar to that of a cell membrane enclosing the contents of a cell. Liposomes can be loaded with active agents, such as lipophilic molecules (e.g., lipophilic active agents) and/or hydrophilic molecules (e.g., hydrophilic active agents). For example, the aqueous (e.g., hydrophilic) internal environment of the liposome can protect encapsulated hydrophilic molecules from physiological degradation. Besides the internal aqueous environment, hydrophobic molecules can also be embedded between the lipid membranes and/or adsorb on the liposome surface. Thus, the lipid bilayer of a liposome has similarities to a cell membrane, e.g., without the protein components present in a cell membrane.

Liposomes may be characterized, e.g., based on their structure, size, shape, membrane type, and surface properties. For example, liposomes can be classified based on their size and number of lipid bilayers (also referred to as “lamellarity”). Liposomes can be classified as unilamellar vesicles (UVs), oligolamellar vesicles (OLVs), multilamellar vesicles (MLVs), and multivesicular vesicles (MVVs) according to their lamellarity. Oligolamellar vesicles (OLVs) typically comprises between about two to about five concentric lipid bilayers. Multivesicular vesicles (MVVs) encapsulate multiple non-concentric bilayer liposomes. MLVs typically comprises more than about five non-concentric bilayer liposomes. Additionally, UVs can be divided into small unilamellar vesicles (SUVs) with a particle size of less than about 100 nm (e.g., <100 nm, e.g., about 1 nm to about 100 nm), large unilamellar vesicles (LUVs) with a particle size of greater than about 100 nm (e.g., >100 nm, e.g., about 100 to about 1000 nm), and giant unilamellar vesicles (GUVs) with a particle size greater than about 1 m (e.g., >1000 nm) according to their size. In some embodiments, oligolamellar vesicles (OLVs) have a particle size of between about 100 to about 1000 nm. In some embodiments, multilamellar large vesicles (MLVs) have a particle size of greater than about 500 nm (e.g., >500 nm). In some embodiments, multivesicular vesicles (MVVs) have a particle size of greater than about 1 m (e.g., >1000 nm). Generally, dynamic light scattering (DLS) and nanoparticle tracking analysis (NTA) techniques can be used to evaluate the size distribution of lipid vesicles. Common methods that can be used to determine the lamellarity of liposomes include nuclear magnetic resonance (NMR), small-angle X-ray scattering (SAXS), and transmission electron microscopy (TEM).

### Lithe Liposomes

The compositions of the present disclosure comprise a lithe liposome. As used herein, the terms “lithe liposome” and “Vecell” refer to liposomes comprising a lipid bilayer, e.g., a phospholipid bilayer, structure modified by one or more lipid bilayer modifiers and/or one or more absorption enhancers. The lithe liposomes of the present disclosure are generally prepared by the addition of one or more lipid bilayer modifier and/or one or more absorption enhancer into a conventional liposome.

As used herein, the term “conventional liposome” refers to any liposome comprising a lipid bilayer, e.g., a phospholipid bilayer, structure that is not modified by a lipid bilayer modifier and/or an absorption enhancer, e.g., as described herein. Although conventional liposomes may provide benefits as a biocompatible and efficient drug delivery platform, e.g., for topical or injectable applications, conventional liposomes have been generally considered of little or no value as carriers for oral drug delivery because of their instability in the gastrointestinal tract and poor permeability across intestinal epithelia. The present disclosure is based, at least in part, on the design and generation of lithe liposome compositions, having, in part, modified lipid bilayers, and improved physicochemical, mechanical, and structural properties to facilitate oral drug delivery. The use of such lithe liposome compositions can improve the oral absorption, oral bioavailability, and/or bio-variability of active agents, e.g., as compared to the use of a reference composition, such as a conventional drug delivery system (e.g., an oil-based composition, a micellized composition, and/or a conventional liposome).

In contrast to conventional liposomes, e.g., for topical or injectable applications, the lithe liposomes described herein are effective for the oral delivery of active agents. In certain embodiments, the lithe liposome compositions described herein can improve the oral bioavailability of an active agent as compared to delivery of the active agent in the absence of the lithe liposome composition, for example, by using a conventional drug delivery system (e.g., an oil-based composition, a micellized composition, and/or a conventional liposome). In certain embodiments, the lithe liposome compositions described herein can improve the oral bioavailability of an active agent by improving the solubility and/or permeability of the active agent.

In certain embodiments, the lithe liposome compositions described herein can improve the oral bioavailability of an active agent by modulating, e.g., increasing, the stability, solubility, and/or permeability of the active agent by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% or more, e.g., as compared to the use of a reference composition, such as a conventional drug delivery system (e.g., an oil-based composition, a micellized composition, and/or a conventional liposome).

In certain embodiments, the lithe liposome compositions described herein can improve the oral bioavailability of an active agent by modulating, e.g., increasing, the stability, solubility, and/or permeability of the active agent by at least about 2-fold, or at least about 3-fold, or at least about 4-fold, or at least about 5-fold, or at least about 10-fold or more, e.g., as compared to the use of a reference composition, such as a conventional drug delivery system (e.g., an oil-based composition, a micellized composition, and/or a conventional liposome).

### Lipids and Phospholipids

In some embodiments, the lithe liposome composition comprises a lipid, such as a phospholipid or a phospholipid derivative. In certain embodiments, a single kind or type of phospholipid may be used in the creation of the lithe liposome compositions. In other embodiments, more than one kind or type of phospholipid may be used.

As used herein, the term “phospholipid” refers to a class of lipids having a hydrophobic tail (e.g., one or two) and a phosphate head group. A phospholipid is generally an amphipathic molecule. The phosphate group is negatively charged, making the head polar and hydrophilic. The lipid tail is uncharged, nonpolar, and hydrophobic. As such, hydrophilicity is conferred to the phospholipid by its phosphate head group and hydrophobicity is conferred to the phospholipid by uncharged, nonpolar groups that include long-chain saturated and unsaturated aliphatic hydrocarbon groups and such groups substituted by one or more aromatic, cycloaliphatic, or heterocyclic groups (e.g., fatty acid acyl groups). In certain embodiments, a phospholipid is a naturally-occurring phospholipid. In other embodiments, a phospholipid is a synthetic phospholipid.

The term “phospholipid” also refers to phosphatidic acids, phosphoglycerides, and phosphosphingolipids. Phosphatidic acids include a phosphate group coupled to a glycerol group, which may be mono- or diacylated. Phosphoglycerides (or glycerophospholipids) include a phosphate group intermediate an organic group (e.g., choline, ethanolamine, serine, inositol) and a glycerol group, which may be mono- or diacylated. Phosphosphingolipids (or sphingomyelins) include a phosphate group intermediate an organic group (e.g., choline, ethanolamine) and a sphingosine (non-acylated) or ceramide (acylated) group.

In certain embodiments, the phospholipids useful in the compositions and methods of the present disclosure can include their salts (e.g., sodium, ammonium). For phospholipids that include carbon-carbon double bonds, individual geometrical isomers (cis, trans) and mixtures of isomers can be included.

Examples of suitable phospholipids include esters of glycerol with one or two (equal or different) residues of fatty adds and with phosphoric acid, wherein the phosphoric acid residue is in turn bound to a hydrophilic group, such a choline (phosphatidylcholines, PC), serine (phosphatidylserines, PS), glycerol (phosphatidylglycerols, PG), ethanolamine (phosphatidylethanolamines, PE), inositol (phosphatidylinositol, PI). Esters of phospholipids with only one residue of fatty acid are generally referred to in the art as the “lyso” forms of the phospholipid or “lysophospholipids.” Fatty acids residues present in the phospholipids are in general long chain aliphatic acids, typically containing from about 12 to about 24 carbon atoms, e.g., from about 14 to about 22 carbon atoms. The aliphatic chain may contain one or more unsaturations or is completely saturated. Examples of suitable fatty acids included in the phospholipids are, for example, lauric acid, myristic acid, palmitic acid, stearic acid, arachidic acid, behenic acid, oleic acid, linoleic acid, and/or linolenic acid. In some embodiments, saturated fatty acids such as myristic acid, palmitic acid, stearic acid, and/or arachidic acid are employed.

Representative phospholipids include phosphatidylcholines, phosphatidylethanolamines, phosphatidylglycerols, phosphatidylserines, phosphatidylinositols, and phosphatidic acids, and their lysophosphatidyl (e.g., lysophosphatidylcholines and lysophosphatidylethanolamine) and diacyl phospholipid (e.g., diacylphosphatidylcholines, diacylphosphatidylethanolamines, diacylphosphatidylglycerols, diacylphosphatidylserines, diacylphosphatidylinositols, and diacylphosphatidic acids) counterparts.

The acyl groups of the phospholipids may be the same or different. In certain embodiments, the acyl groups are derived from fatty acids having C₁₀-C₂₄ carbon chains (e.g., acyl groups such as decanoyl (C10), dodecanoyl (also known as lauroyl) (C12), tetradecanoyl (also known as myristoyl) (C14), hexadecanoyl (also known as palmitoyl) (C16), octadecanoyl (also known as stearoyl) (C18), oleoyl, linoleoyl, linolenoyl, arachidonoyl groups).

Representative diacylphosphatidylcholines (e.g., 1,2-diacyl-sn-glycero-3-phosphocholines) include distearoylphosphatidylcholine (DSPC), dioleoylphosphatidylcholine (DOPC), dipalmitoylphosphatidylcholine (DPPC), dilinoleoylphosphatidylcholine (DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), didodecanoylphosphatidylcholine, dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoylphosphatidylcholine (SMPC), palmitoylmyristoylphosphatidylcholine (PMPC), palmitoylstearoylphosphatidylcholine (PSPC), stearoylpalmitoylphosphatidylcholine (SPPC), and stearoyloleoylphosphatidylcholine (SOPC).

Representative diacylphosphatidylethanolamines (e.g., 1,2-diacyl-sn-glycero-3-phosphoethanolamines) include dioleoylphosphatidylethanolamine (DOPE), dipalmitoylphosphatidylethanolamine (DPPE), distearoylphosphatidylethanolamine (DSPE), dilauroylphosphatidylethanolamine (DLPE), dimyristoylphosphatidylethanolamine (DMPE), dierucoylphosphatidylethanolamine (DEPE), didecanoylphosphatidylethanolamine, didodecanoylphosphatidylethanolamine, and palmitoyloleoylphosphatidylethanolamine (POPE).

Representative diacylphosphatidylglycerols (e.g., 1,2-diacyl-sn-glycero-3-phosphoglycerols) include dioleoylphosphatidylglycerol (DOPG), dipalmitoylphosphatidylglycerol (DPPG), dierucoylphosphatidylglycerol (DEPG), dilauroylphosphatidylglycerol (DLPG), dimyristoylphosphatidylglycerol (DMPG), distearoylphosphatidylglycerol (DSPG), didecanoylphosphatidylglycerol, didodecanoylphosphatidylglycerol, and palmitoyloleoylphosphatidylglycerol (POPG).

Representative diacylphosphatidylserines (e.g., 1,2-diacyl-sn-glycero-3-phosphoserines) include dilauroylphosphatidylserine (also known as didodecanoylphosphatidylserine) (DLPS), dioleoylpho sphatidylserine (DOPS), dipalmitoylphosphatidylserine (DPPS), didecanoylpho sphatidylserine, and distearoylphosphatidylserine (DSPS).

Representative diacylphosphatidic acids (e.g., 1,2-diacyl-sn-glycero-3-phosphates) include dierucoylphosphatidic acid (DEPA), dilauroylphosphatidic acid (also known as didodecanoylphosphatidic acid) (DLPA), dimyristoylphosphatidic acid (DMPA), dioleoylphosphatidic acid (DOPA), dipalmitoylphosphatidic acid (DPPA), didecanoylphosphatidic acid, and distearoylphosphatidic acid (DSPA).

Representative phospholipids include phosphosphingolipids such as ceramide phosphoryllipid, ceramide phosphorylcholine, and ceramide phosphorylethanolamine.

As used herein, the term phospholipids include either naturally occurring, semisynthetic or synthetically prepared products that can be employed either singularly or as mixtures.

Examples of naturally occurring phospholipids are natural lecithins (phosphatidylcholine (PC) derivatives) such as, typically, soya bean, sunflower or egg yolk lecithins.

Examples of semisynthetic phospholipids are the partially or fully hydrogenated derivatives of the naturally occurring lecithins. In some embodiments, the phospholipids are fatty acids di-esters of phosphatidylcholine, ethylphosphatidylcholine, phosphatidylglycerol, phosphatidic acid, phosphatidylethanolamine, phosphatidylserine or of sphingomyelin. Examples of preferred phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG), diarachidoylphosphatidylglycerol (DAPG), dimyristoylphosphatidylglycerol (DMPG), dipalmitoylphosphatidylglycerol (DPPG), distearoylphosphatidylglycerol (DSPG), dioleoyl-phosphatidylglycerol (DOPG), dimyristoyl phosphatidic acid (DMPA), dipalmitoyl phosphatidic acid (DPPA), distearoyl phosphatidic acid (DSPA), diarachidoylphosphatidic acid (DAPA), dimyristoylphosphatidylethanolamine (DMPE), dipalmitoylphosphatidylethanolamine (DPPE), distearoyl phosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI).

The term phospholipid further includes modified phospholipid, e.g., phospholipids where the hydrophilic group is in turn bound to another hydrophilic group. Examples of modified phospholipids are phosphatidylethanolamines modified with polyethylenglycol (PEG), e.g., phosphatidylethanolamines where the hydrophilic ethanolamine moiety is linked to a PEG molecule of variable molecular weight (e.g., from 300 to 5000 daltons), such as DPPE-PEG (or DSPE-PEG), e.g., DPPE (or DSPE) having a PEG polymer attached thereto. For example, DPPE-PEG2000 refers to DPPE having attached thereto a PEG polymer having a mean average molecular weight of about 2000.

Examples of phospholipids bearing an overall negative charge are derivatives, in particular fatty acid di-ester derivatives, of phosphatidylserine, such as DMPS, DPPS, DSPS; of phosphatidic acid, such as DMPA, DPPA, DSPA; of phosphatidylglycerol such as DMPG, DPPG and DSPG or of phosphatidylinositol, such as DMPI, DPPI or DPPI. Also modified phospholipids, in particular PEG-modified phosphatidylethanolamines, such as DMPE-PEG2000, DMPE-PEG3000, DMPE-PEG4000, DPPE-PEG5000, DPPE-PEG2000, DPPE-PEG3000, DPPE-PEG4000, DPPE-PEG5000, DSPE-PEG2000, DSPE-PEG3000, DSPE-PEG4000, DSPE-PEG5000, DAPE-PEG2000, DAPE-PEG3000, DAPE-PEG4000 or DAPE-PEG5000 can be used as negatively charged molecules. Also the lyso-form of the above cited phospholipids, such as lysophosphatidylserine derivatives (e.g., lyso-DMPS, -DPPS or -DSPS), lysophosphatidic add derivatives (e.g., lyso-DMPA, -DPPA or -DSPA) and lysophosphatidylglycerol derivatives (e.g., lyso-DMPG, -DPPG or -DSPG), can advantageously be used as negatively charged compound. Examples of negatively charged lipids are bile acid salts such as cholic acid salts, deoxycholic acid salts or glycocholic acid salts; and (C12-C24), preferably (C14-C22) fatty acid salts such as, for instance, palmitic acid salt, stearic acid salt, 1,2-dipalmitoyl-sn-3-succinylglycerol salt or 1,3-dipalmitoyl-2-succinylglycerol salt.

Examples of phospholipids bearing an overall positive charge are derivatives of ethylphosphatidylcholine, in particular di-esters of ethylphosphatidylcholine with fatty acids, such as 1,2-Distearoyl-sn-glycero-3-Ethylphosphocholine (Ethyl-DSPC or DSEPC), 1,2-Dipalmitoyl-sn-glycero-3-Ethylphosphocholine (Ethyl-DPPC or DPEPC). The negative counterion is preferably an halogen ion, in particular chlorine or bromine. Examples of positively charged lipids are alkylammonium salts with a halogen counter ion (e.g., chlorine or bromine) comprising at least one (C10-C20), preferably (C14-C18), alkyl chain, such as, for instance mono or di-stearylammonium chloride, mono or di-hexadecylammonium chloride, dimethyldioctadecylammonium bromide (DDAB), hexadecyltrimethylammonium bromide (CrAB). Further examples of positively charged lipids are tertiary or quaternary ammonium salts with a halogen counter ion (e.g., chlorine or bromine) comprising one or preferably two (C10-C20), preferably (C14-C18), acyl chain linked to the N-atom through a (C3-C6) alkylene bridge, such as, for instance, 1,2-distearoyl-3-trimethylammonium-propane (DSTAP), 1,2-dipalmitoyl-3-trimethylammonium-propane (DPTAP), 1,2-oleoyl-3-trimethylammonium-propane (DOTAP), 1,2-distearoyl-3-dimethylammonium-propane (DSDAP).

In some embodiments, the lithe liposome composition comprises about ten or fewer types of phospholipids, or about five or fewer types of phospholipids, or about three or fewer types of phospholipids.

In some embodiments, the molar percentage (mol %) of a specific type of phospholipid present typically comprises from about 0% to about 10%, from about 10% to about 30%, from about 30% to about 50%, from about 50% to about 70%, from about 70% to about 90%, from about 90% to 100% of the total phospholipid present in a liposome.

In some embodiments, the lithe liposome composition comprises at least about 0.01% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 2% w/v, about 3% w/v, about 4% w/v, about 5% w/v, about 6% w/v, about 7% w/v, about 8% w/v, about 9% w/v, about 10% w/v, about 15% w/v, about 20% w/v, about 25% w/v, about 30% w/v, about 35% w/v, about 40% w/v, about 45% w/v, about 50% w/v, about 55% w/v, about 60% w/v, about 65% w/v, about 70% w/v, about 75% w/v, about 80% w/v, about 85% w/v, about 90% w/v, about 95% w/v or more) of a phospholipid, e.g., as described herein.

In some embodiments, the lithe liposome composition comprises at least about 0.01% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 2% w/v, about 3% w/v, about 4% w/v, about 5% w/v, about 6% w/v, about 7% w/v, about 8% w/v, about 9% w/v, about 10% w/v, about 15% w/v, about 20% w/v, about 25% w/v, about 30% w/v, about 35% w/v, about 40% w/v, about 45% w/v, about 50% w/v, about 55% w/v, about 60% w/v, about 65% w/v, about 70% w/v, about 75% w/v, about 80% w/v, about 85% w/v, about 90% w/v, about 95% w/v or more) of a phosphatidylcholine or a derivative thereof.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a phospholipid, e.g., as described herein.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a phosphatidylcholine or a derivative thereof.

In some embodiments, the lithe liposome composition comprises about 0.1% w/v to about 5% w/v (e.g., about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, or about 5% w/v) of a phosphatidylcholine or a derivative thereof.

In some embodiments, the lithe liposome composition comprises about 0.01% w/v to about 1% w/v (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.11% w/v, about 0.12% w/v, about 0.13% w/v, about 0.14% w/v, about 0.15% w/v, about 0.16% w/v, about 0.17% w/v, about 0.18% w/v, about 0.19% w/v, about 0.2% w/v, about 0.21% w/v, about 0.22% w/v, about 0.23% w/v, about 0.24% w/v, about 0.25% w/v, about 0.26% w/v, about 0.27% w/v, about 0.28% w/v, about 0.29% w/v, about 0.3% w/v, about 0.31% w/v, about 0.32% w/v, about 0.33% w/v, about 0.34% w/v, about 0.35% w/v, about 0.36% w/v, about 0.37% w/v, about 0.38% w/v, about 0.39% w/v, about 0.4% w/v, about 0.41% w/v, about 0.42% w/v, about 0.43% w/v, about 0.44% w/v, about 0.45% w/v, about 0.46% w/v, about 0.47% w/v, about 0.48% w/v, about 0.49% w/v, about 0.5% w/v, about 0.51% w/v, about 0.52% w/v, about 0.53% w/v, about 0.54% w/v, about 0.55% w/v, about 0.56% w/v, about 0.57% w/v, about 0.58% w/v, about 0.59% w/v, about 0.6% w/v, about 0.61% w/v, about 0.62% w/v, about 0.63% w/v, about 0.64% w/v, about 0.65% w/v, about 0.66% w/v, about 0.67% w/v, about 0.68% w/v, about 0.69% w/v, about 0.7% w/v, about 0.71% w/v, about 0.72% w/v, about 0.73% w/v, about 0.74% w/v, about 0.75% w/v, about 0.76% w/v, about 0.77% w/v, about 0.78% w/v, about 0.79% w/v, about 0.8% w/v, about 0.81% w/v, about 0.82% w/v, about 0.83% w/v, about 0.84% w/v, about 0.85% w/v, about 0.86% w/v, about 0.87% w/v, about 0.88% w/v, about 0.89% w/v, about 0.9% w/v, about 0.91% w/v, about 0.92% w/v, about 0.93% w/v, about 0.94% w/v, about 0.95% w/v, about 0.96% w/v, about 0.97% w/v, about 0.98% w/v, about 0.99% w/v, or about 1% w/v) of a phosphatidylcholine or a derivative thereof.

### Cholesterol

In some embodiments, the lithe liposome composition comprises a sterol or a derivative thereof, optionally wherein the sterol or the derivative thereof comprises a cholesterol or a derivative thereof.

In some embodiments, the lithe liposome composition comprises a cholesterol or a cholesterol derivative. In other embodiments, the lithe liposome composition is substantially free of cholesterol or a derivative thereof.

As used herein, the term “cholesterol derivative” refers to a derivative of the molecule cholesterol. Representative, non-limiting examples of cholesterol derivatives include ldosterone, beclomethasone, betamethasone, cholesterol, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, oxandrolone, oxymetholone, paramethasone, prednisolone, prednisone, stanozolol, and triamicinolone, testosterone, dehvdroeniandrosterone, androstenedione, dihydrotestosterone, aldosterone, estradiol, estrone, estriol, cortisol, oroaesterone and hydroxy cholesterol.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a sterol or a derivative thereof, e.g., as described herein.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a cholesterol or a cholesterol derivative, e.g., as described herein.

### Lipid Bilayer Modifier

In some embodiments, the lithe liposome composition comprises a lipid bilayer modifier.

As used herein, the term “lipid bilayer modifier” refers to a surfactant, a solvent, a polysaccharide e.g., a compound capable of causing the destabilization of the lipid bilayer of the vesicle and increases the vesicle-elasticity or fluidity by lowering its interfacial tension. In some embodiments, the lipid bilayer modifier is selected from the group consisting of a surfactant, a solvent, a polysaccharide, and combinations thereof.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a lipid bilayer modifier, e.g., as described herein.

### (i) Surfactants

In some embodiments, the lipid bilayer modifier comprises a surfactant.

As used herein, the term “surfactant” refers to surface active agents. Surfactants are generally amphiphilic compounds which decrease surface or interfacial tensions. The term “surfactant” includes reference to all forms of the surfactant, including surfactant salts, and reference to the weight percentage of a surfactant includes the complete weight of the specified form such as chelants, sometimes referred to as chelators or sequestrants, and/or the counterion of any surfactant salts.

In some embodiments, the lipid bilayer modifier comprises an anionic surfactant. As used herein, the term “anionic surfactant” refers to a surfactant wherein the hydrophilic or polar group has an anionic charge. Exemplary anionic surfactants include sulfates, such as sodium alkyl sulfate, sodium dodecyl sulfate, sodium dodecylbenzenesulfonate, sodium laurate, sodium laureth sulfate, sodium lauryl sarcosinate, potassium lauryl sulfate, ammonium lauryl sulfate, ammonium laureth sulfate, ammonium xylene sulfonate, magnesium laureth sulfate, and sodium myreth sulfate; sulfonates, such as sodium nonanoyloxybenzenesulfonate; carboxylates; sulphated esters; sulphated alkanolamides; alkylphenols; and combinations thereof.

In some embodiments, the lipid bilayer modifier comprises a non-ionic surfactant. As used herein, the term “non-ionic surfactant” refers to a surfactant wherein the hydrophilic or polar group does not have an ionic charge. Exemplary nonionic surfactants include fatty alcohols such as cetyl alcohol, stearyl alcohol, cetostearyl alcohol, and oleyl alcohol, polyoxamers, ethoxylated fatty alcohols, such as PEG-80 sorbitan laurate, polyoxyethylene glycol alkyl ethers, such as octaethylene glycol monododecyl ether, and pentaethylene glycol monododecyl ether, polyoxypropylene glycol alkyl ethers, glucoside alkyl ethers, polyoxyethylene glycol octylphenol ethers, polyoxyethylene glycol alkylphenol ethers, such as nonoxynol-9, glycerol alkyl esters such as glyceryl laurate, polyoxyethylene glycol sorbitan alkyl esters, such as polysorbate, sorbitan alkyl esters, cocamide MEA, cocamide DEA, amine oxides, such as dodecyldimethylamine oxide, block copolymers of polyethylene glycol and polypropylene glycol, such as poloxamers, polyethoxylated tallow amine, and combinations thereof. In some embodiments, the non-ionic surfactant is a polysorbate or a poloxamer. In some embodiments, the polysorbate is polysorbate 20, polysorbate 40, polysorbate 60 or polysorbate 80.

In some embodiments, the lipid bilayer modifier comprises a cationic surfactant. As used herein, the term “cationic surfactant” refers to a surfactant wherein the hydrophilic or polar group has a cationic charge. Exemplary cationic surfactants include quaternary ammonium salts, linear alkyl-amines, and alkyl ammoniums. In some embodiments, the cationic surfactant comprises cetylpyridinium chloride.

In some embodiments, the lipid bilayer modifier comprises a zwitterionic surfactant. As used herein, the term “zwitterionic surfactant” refers to a surfactant which has in its structure one or more negative charges and one or more positive charges, these being balanced to give a total charge of zero. Exemplary zwitterionic surfactants include betaines, such as cocamidopropyl betaine; sultaines, such as cocamidopropyl hydroxyl sultaine and lauramidopropyl hydroxyl sultaine; and amphoacetates and amphodiacetates, such as disodium lauroamphodiacetate, disodium cocoamphodiacetate, sodium lauroamphoacetate, sodium cocoamphoacetate, disodium cocoamphodipropionate and disodium lauroamphodipropionate; and combinations thereof.

In some embodiments, the lipid bilayer modifier comprises a single chain surfactant.

In some embodiments, the lipid bilayer modifier comprises bile acids and/or bile salts. Bile acids, also known as steroid acids, are amphiphilic water-soluble molecules that can be derived from cholesterol. In the body, bile acids in bile may be conjugated with glycine or taurine to form bile salts to increase their water solubility. However, in concentrations above the critical micellar concentration (CMC), bile acids can solubilize lipid layers and wedge between fatty acid molecules to form micelles. This ability of bile salt may account for the detergent properties of bile. One of skill in the art would appreciate that in the body, bile acids can be important to the formation of both bile and intestinal micelles, which can facilitate the transport of insoluble cholesterol from the liver to the intestine and for the transport of digested fats within the intestine to enterocytes for absorption, but do not have a bilayer structure. The primary bile acids cholic acid (CA) and chenodeoxycholic acid (CDCA) can be synthesized in the liver and, in some instances, may be conjugated with glycine or taurine by the enzymes BA-CoA synthase (BACS) and BA-amino acid transferase (BAT) in the liver to produce glyco-CA (glycocholic acid, GCA), tauro-CA (taurocholic acid, TCA), glyco-CDCA (glycochenodeoxycholic acid, GCDCA), and/or tauro-CDCA (taurochenodeoxycholic acid, TCDCA) bile acids. In the intestine, the primary bile acids (e.g., CA and/or CDCA) and/or the primary conjugated bile acids (e.g., GCA, GCDCA, TCA, and/or TCDCA) may be modified by bacteria, e.g., by bacterial enzymes, to produce secondary bile acids, such as deoxycholic acid (DCA) and lithocholic acid (LCA). Bile salts are naturally occurring rigid surfactants with a steroidal skeleton and specific self-assembly and interface behaviors. In some embodiments, “bile salts” may be formed by the conjugation of bile acids (e.g., CA and/or CDCA) with an amino acid (e.g., glycine and/or taurine).

In some embodiments, the lipid bilayer modifier is selected from the group consisting of a polysorbate 20, a polysorbate 60, and a polysorbate 80, a span 20, a span 40, a span 60, a span 80, a bile salt, and a combination thereof.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a surfactant, e.g., as described herein. In some embodiments, the surfactant comprises Span 60. In some embodiments, the surfactant comprises polysorbate 80. In some embodiments, the surfactant comprises sodium cholate. In some embodiments, the surfactant comprises a bile salt.

### (ii) Solvents

In some embodiments, the lipid bilayer modifier comprises a solvent.

As used herein, the term “solvent” refers to any substance or liquid capable of capable completely or partially dissolving, dispersing, or suspending one or more other substances, e.g., a lithe liposome as described herein, into solution.

Exemplary solvents include, but are not limited to, acetone, acetonitrile, dioxane, ethanol, 2-methoxy ethyl acetate, methoxy ethanol, ethoxy ethanol, butoxy ethanol, 2-propanol, propylene glycol methyl ether, ethanediol, 1,2-propanediol, tert-butyl alcohol, di ethylene glycol, methanol (e.g., L-menthol), N-methylpyrrolidone, dimethylacetamide, dimethylformamide, dimethylsulfoxide, pyridine, tetrahydrofuranacetonitrile, and combinations thereof. In some embodiments, the lipid bilayer modifier comprises ethanol.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a solvent, e.g., as described herein.

In some embodiments, the solvent comprises ethanol.

(iii) Polysaccharides

In some embodiments, the lipid bilayer modifier comprises a polysaccharide.

As used herein, the term “polysaccharide” refers to carbohydrate molecules of repeated monomer (monosaccharide) units joined together by glycosidic bonds. The polysaccharide may vary in structure, for example, may be linear or branched. The molecules may contain modifications of the repeating unit. Monosaccharides are generally aldehydes or ketones with two or more hydroxyl groups. A polysaccharide containing a single type of monosaccharide unit is referred to as a homopolysaccharide, while a polysaccharide containing more than one type of monosaccharide unit is referred to as a heteropolysaccharide. Polysaccharides are generally considered to contain ten or more monosaccharide units, while the term “oligosaccharide” is generally used to refer to the polymers containing a small number, e.g., two to ten, of monosaccharide units.

Exemplary polysaccharides include, but are not limited to, starches, modified starches, glucans, dextrins, cyclodextrins, celluloses, modified celluloses, hemicelluloses, pectins, chitins, chitosans, gums, lignin, modified gums, and combinations thereof. In some embodiments, the polysaccharide comprises cyclodextrin.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a polysaccharide, e.g., as described herein. In some embodiments, the polysaccharide comprises cyclodextrin. In some embodiments, the polysaccharide comprises a cyclic oligosaccharide comprising 6, 7, or 8 glucopyranose units, and which may be referred to as α-, β-, or γ-cyclodextrins, respectively.

In some embodiments, the polysaccharide comprises a hydroxypropyl-β-cyclodextrin (HBC) or a derivative thereof.

### Absorption Enhancers

In some embodiments, the lithe liposome composition comprises an absorption enhancer. As used herein, the term “absorption enhancer” refers to a functional excipient included in compositions to improve the absorption of an active agent. The term “absorption enhancer” generally refers to an agent whose function is to increase absorption by enhancing mucous membrane permeation, rather than increasing solubility. As such, such agents are sometimes referred to as “permeation enhancers.” In particular, absorption enhancers described herein may improve paracellular transport (e.g., passage through intercellular spaces and tight junctions), transcellular transport (e.g., passive diffusion or active transport across cellular membranes), and/or transcytosis (e.g., cellular vesicular uptake).

In some embodiments, the absorption enhancer comprises a polyphenol. In some embodiments, the absorption enhancer comprises a flavonoid, such as quercetin or dihydroquercetin.

In some embodiments, the absorption enhancer comprises a terpene, such as menthol.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of an absorption enhancer, e.g., as described herein.

### (i) Terpenes

In some embodiments, the lithe liposome composition comprises a terpene. As used herein, the term “terpene” refers to a class of compounds derived from isoprene, which has the molecular formula C₅H₈. As used herein, the term “terpene” can refer to both terpenes and terpenoids. The basic molecular formula for terpenes comprises multiples of C₅H₈, that is, (C₅H₈)ₙ where n is the number of linked isoprene units. The isoprene units may be linked together head to head to form linear chains, or they may be arranged to form rings. Terpenes may be classified by the number of isoprene units (n≥2) in the molecule. Hemiterpenes comprise a single isoprene unit and have five carbon atoms. Monoterpenes comprise two isoprene units and are represented by the formula C₁₀H₁₆. Examples of monoterpenes and monoterpenoids include, without limitation, geraniol, terpineol, limonene, myrcene, linalool, hinokitiol, pinene, camphor, menthol, carvone, terpineol, and thujone. Iridoids can be derived from monoterpenes. Examples of iridoids include, without limitation, aucubin and catalpol. Sesquiterpenes comprise three isoprene units and are represented by the formula C₁₅H₂₄. Examples of sesquiterpenes and sesquiterpenoids include, without limitation, humulene, farnesenes, farnesol, and geosmin. Diterpenes comprise four isoprene units and are represented by the formula C₂₀H₃₂. Examples of diterpenes and diterpenoids include, without limitation, cafestol, kahweol, cembrene, and taxadiene. Sesterterpenes comprise five isoprene units and have 25 carbon atoms. An example of a sesterterpenoid includes, without limitation, geranylfarnesol. Triterpenes comprise six isoprene units and are represented by the formula C₃₀H₄₈. An example of a triterpenes includes, without limitation, squalene. Sesquarterpenes comprise seven isoprene units and have the molecular formula C₃₅H₅₆. Examples of sesquarterpenoids include, without limitation, ferrugicadiol and tetraprenylcurcumene. Tetraterpenes comprise eight terpene units and are represented by the formula C₄₀H₆₄. Examples of tetraterpenoids include, without limitation, acylic lycopene, monocyclic gamma-carotene, alpha-carotene, and beta-carotene. Polyterpenes comprise long chains of many isoprene units. Norisoprenoids are characterized by the shortening of a chain or ring by the removal of a methylene group or substitution of one or more methyl side chains by hydrogen atoms. Examples of norisoprenoids include, without limitation, C₁₃-norisoprenoid 3-oxo-α-ionol and 7,8-dihydroionone derivatives, such as megastigmane-3,9-diol and 3-oxo-7,8-dihydro-α-ionol.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a terpene, e.g., as described herein.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of menthol.

### (ii) Polyphenols

In some embodiments, the lithe liposome composition comprises a polyphenol.

As used herein, the term “polyphenols” refers to a compound containing more than one phenolic hydroxyl group. Polyphenols are a structural class of mainly natural, but also synthetic or semisynthetic organic chemicals characterized by the presence of large multiples of phenol structural units.

Exemplary polyphenols include, without limitation, dopamine, adrenaline, noradrenaline, salbutamol, curcuminoids and/or its derivatives, yakuchinone A, yakuchinone B, rosmarinic acid, paradol, hydroxytyrosol, silymarin, coumarin, esculetin, escopoletin, lignans (including sesamol, sesamin, sesamolin), carnosol, oleuropein, uric acid, ubiquinol, thymolphtaleine, phenolphthalein, carthamin, polyporic acid, atromentin, bovichinon-3, grevillin A, grevillin B, grevillin D, alkannin, shikonin, alizarin, purpurin, pseudopurpurin, purpuroxanthin, rubiadin, munjistin, chinizarin, morindon, emodin, aloe-emodin, rhein, chrysophanol, kermesic acid, flavokermesic acid, carminic acid, ellagic acid, spinochrome C, spinochrome D, spinochrome E, echinochrome A, red alkannin, hypericin, chrysophanic acid, betanidin, isobetanidin, pyrocatechol, pyrogallol, gallic acid and/or its esters, caftaric acid, chlorogenic acid, elonolic acid, protocatechuic acid, syringic acid, gentisic acid, caffeic acid, hops acids (including humulone, lupulone, colupulone), magnolol, honokiol, biphenols, di-resorcinol sulphide, bithionol, bromochlorophen, dioxybenzone, bisoctrizole, bemotrizinol, resveratrol, tannins (such as tannic acid), phenylpropanoids, flavonoids (including flavones (such as luteolin, apigenin, baicalin, tangeritin), flavonols (such as quercetin, galantin, kaempferol, myricetin, fisetin, isorhamnetin, pachypodol, rhamnazin, rutin, hydroxyethylrutosides), flavanones (such as hesperetin, naringenin, eriodictyol), 3-hydroxyflavanones (such as dihydroquercetin, dihydrokaempferol), isoflavones (such as genistein, daidzein, glycitein), neoflavonoids, flavan-3-ols (such as catechins, theaflavins), and anthocyanidins (such as cyanidin, delphinidin, malvidin, pelargonidin, peonidin, petunidin)), inclusive of variants or derivatives thereof.

Polyphenols include, for example, constituents of the subgroup of flavonoids, which includes, without limitation, isoflavonoids, flavanones, flavanols, flavonols, flavones, and anthocyanidins Exemplary flavonoids include, but are not limited to, quercetin, rutin, luteolin-7-O-β-D-glucopyranoside, kaempferol-3-O-β-D-glucopyranoside, apigenin-7-O-α-L-rhamnopyranoside, chrysoeriol-7-O-β-D-glucopyranosyl, luteolin-3′-L-rhamnoside, luteolin, flavoyadorinin-B, rhoifolin, quercetin-3-O-β-D-glucopyranoside, 3′-methoxy luteolin, 5,3′-dimethoxy luteolin, luteolin-5-O-β-D-glucopyranoside, apigenin, isorhamnetin-3-O-β-D-glucopyranoside, hyperoside, quercetin-7-O-β-D-glucopyranoside, kaempferol-3-O-β-D-rutinoside, isorhamnetin-3-O-β-D-rutinoside, 5-hydroxyl-3′,4′,7-trimethoxy flavone, 5-hydroxyl-6,7,8,4′-tetramethoxy flavone, corymbosin 5-hydroxyl-7,4′-dimethoxy flavone, lonicerin, 5,7,3′,4′,5′-pentamethoxy flavone, and 5,4′-dihydroxy-3′,5′-dimethoxy-7-β-D-glucoxy-flavone.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a polyphenol, e.g., as described herein.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of a flavonoid, e.g., as described herein.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of quercetin.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of dihydroquercetin.

### Active Agents

In some embodiments, the lithe liposome composition comprises an active agent.

As used herein, the term “agent,” “active agent,” or “therapeutic agent” refers to any biological and/or chemical entity that has certain function or activity. An active agent includes, but is not limited to, an atom, a chemical group, a small molecule, an organic compound, an inorganic compound, a nucleoside, a nucleotide, a nucleobase, a sugar, a nucleic acid, an amino acid, a peptide, a polypeptide, a protein, a fusion protein, a protein complex, a dietary supplement, or a drug. The agent may be detected by methods known in the art. The function or activity of an agent may include any physical, chemical, biological, or physiological function or activity. In certain embodiments, the term “active agent” or “therapeutic agent” refers to any agent that, when administered to a subject, has a therapeutic, prophylactic, and/or diagnostic effect and elicits a desired biological and/or pharmacological effect. In certain embodiments, the term “agent” refers to any substance which one wishes to encapsulate in, attach to or combine with the lithe liposomes of the present disclosure.

In some embodiments, the lithe liposome composition comprises more than one active agent. In some embodiments, the lithe liposome composition comprises a plurality of active agents.

As used herein, the term “plurality” encompasses more than one. In some embodiments, a plurality of active agents refers to at least two active agents, for example, at least 3, at least 4, at least 5, at least 6, at least 7, at least 8, at least 9, at least 10, or more active agents, e.g., as described herein. In one embodiment, each active agent in a plurality may be the same or may be different. In some embodiments, the lithe liposome composition comprises a plurality of active agents (e.g., more than one active agent, e.g., at least two active agents). In some embodiments, each active agent in a plurality of active agents is independently present in an amount of about 0.01% w/v to about 50% w/v. In such embodiments, the amount of active agent in a plurality of active agents may be greater than about 50% w/v.

The active agent can be selected from a variety of known classes of drugs, including, for example, proteins, peptides, nucleotides, anti-obesity drugs, nutriceuticals, cannabinoids, plant alkaloids, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

In some embodiments, the active agent may be selected from the group consisting of a fat-soluble vitamin, a curcuminoid, a cannabidiol (CBD), a berberine, derivatives thereof, salts thereof, and combinations thereof.

In some embodiments, the active agent may be characterized by one or more of the following: (i) low solubility, high permeability; (ii) high solubility, low permeability; (iii) low solubility, low permeability; (iv) low bioavailability; (v) moderate to severe side effects; (iv) instability; and combinations thereof. In one embodiment, the active agent comprises a class of the biopharmaceutical classification system (BCS). In one embodiment, a BCS class is selected from the group consisting of Class I (High permeability, High solubility); Class II (Low solubility, Low Permeability); Class III (High Solubility, Low Permeability); or Class IV (Low solubility, Low permeability). In some embodiments, the active agent is characterized according to a biopharmaceutical classification system (BCS) as set forth in Table 1, optionally wherein the active agent is selected from the group consisting of (i) a Class I drug, optionally characterized by an apparent permeability (Papp) of greater than about 10⁻⁵ (cm/sec) and a dose/solubility (Q) of less than or equal to about 0.5; (ii) a Class II drug, optionally characterized by an Papp of greater than about 10⁻⁵ (cm/sec) and a Q of greater than about 1.0; (iii) a Class III drug, optionally characterized by an Papp of less than about 2×10⁻⁶ (cm/sec) and a Q of less than or equal to about 0.5; and (iv) a Class IV drug, optionally characterized by an Papp of less than about 2×10⁻⁶ (cm/sec) and a Q of greater than about 1.0.

In some embodiments, the term “loaded” or “loading” as used in the context of a “loaded lithe liposome” encompasses a lithe liposome comprising one or more components, such as one or more active agents, that are either, for example: (1) encapsulated inside the lithe liposome (e.g., entirely contained within the lumen of the lithe liposome); (2) associated with and/or partially embedded within the lipid bilayer of the lithe liposome (e.g., partly protruding inside the interior of the lithe liposome, and/or partly protruding outside of the lithe liposome); (3) associated with and/or bound to the outer portion of the lipid bilayer of the lithe liposome or a component thereof (e.g., partly protruding from the lipid bilayer of the lithe liposome and/or fully outside the lithe liposome); or (4) entirely disposed within the lipid bilayer of the lithe liposome (e.g., entirely contained within the lipid bilayer).

In some embodiments, the term “loading” encompasses the process of loading, adding, or including one or more components, such as one or more active agents, to the lithe liposome such that any one or more of the above (1)-(4) resultant loaded lithe liposomes is generated. Accordingly, in some embodiments, the active agent is encapsulated inside the lithe liposome (e.g., entirely contained within the lumen of the lithe liposome). In some embodiments, the active agent is associated with and/or partially embedded within the lipid bilayer of the lithe liposome (e.g., partly protruding inside the interior of the lithe liposome, and/or partly protruding outside of the lithe liposome). In some embodiments, the active agent is associated with and/or bound to the outer portion of the lipid bilayer or a component thereof (e.g., partly protruding from the lipid bilayer of the lithe liposome and/or fully outside the lithe liposome). In some embodiments, the active agent is associated with and/or bound to a component of the lipid bilayer. In some embodiments, the active agent is entirely disposed within the lipid bilayer of the lithe liposome (e.g., entirely contained within the lipid bilayer). In some embodiments, one or more active agents are present on the interior or internal surface of the lithe liposome. In some embodiments, one or more active agents are present on the exterior or external surface of the lithe liposome. In some embodiments, the one or more active agents present on the internal and/or external surface of the lithe liposome are associated with the lithe liposome (e.g., attached to the lipid bilayer or a component thereof) via a chemical interaction selected from the group consisting of, e.g., a bond, such as a covalent bond, a noncovalent bond, an ionic bond, and a hydrogen bond; an electrostatic interaction; a hydrophobic interaction; a van der Waals interaction; a linkage; and combinations thereof. In some embodiments, the one or more active agents present on the internal and/or external surface of the lithe liposome are not associated with the lithe liposome (e.g., the active agent is not attached to the lipid bilayer or a component thereof).

Entrapment efficiency (% EE) is an important parameter to calculate the amount of active agent incorporated into the liposomes and can be expressed as the percentage of active agent encapsulated in liposomes relative to the total amount of active agent. In some embodiments, the lithe liposome compositions are characterized by an encapsulation efficiency (% EE) of at least about 50% (e.g., about 50%, about 55%, about 60%, about 65%, about 70%, about 75%, about 80%, about 85%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.5%, or more).

Drug loading (% DL) is the process of incorporating the active agent into the liposomes. It can be calculated as the percentage of active agent encapsulated in liposomes relative to the total amount of phospholipids used. In some embodiments, the lithe liposome compositions are characterized by a drug loading capacity (% DL) of at least about 10% (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.5%, or more).

In some embodiments, the lithe liposome compositions are characterized by an active agent content (mg/mL) of about 0.1 mg/mL to about 25 mg/mL (e.g., about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, or about 25 mg/mL).

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of an active agent.

In some embodiments, the lithe liposome composition comprises about 1 ng to about 2000 ng (e.g., about 1 ng, about 2 ng, about 3 ng, about 4 ng, about 5 ng, about 6 ng, about 7 ng, about 8 ng, about 9 ng, about 10 ng, about 11 ng, about 12 ng, about 13 ng, about 14 ng, about 15 ng, about 16 ng, about 17 ng, about 18 ng, about 19 ng, about 20 ng, about 21 ng, about 22 ng, about 23 ng, about 24 ng, about 25 ng, about 50 ng, about 75 ng, about 100 ng, about 125 ng, about 150 ng, about 175 ng, about 200 ng, about 225 ng, about 250 ng, about 275 ng, about 300 ng, about 325 ng, about 350 ng, about 375 ng, about 400 ng, about 425 ng, about 450 ng, about 475 ng, about 500 ng, about 525 ng, about 550 ng, about 575 ng, about 600 ng, about 625 ng, about 650 ng, about 675 ng, about 700 ng, about 725 ng, about 750 ng, about 775 ng, about 800 ng, about 825 ng, about 850 ng, about 875 ng, about 900 ng, about 925 ng, about 950 ng, about 975 ng, about 1000 ng, about 1025 ng, about 1050 ng, about 1075 ng, about 1100 ng, about 1125 ng, about 1150 ng, about 1175 ng, about 1200 ng, about 1225 ng, about 1250 ng, about 1275 ng, about 1300 ng, about 1325 ng, about 1350 ng, about 1375 ng, about 1400 ng, about 1425 ng, about 1450 ng, about 1475 ng, about 1500 ng, about 1525 ng, about 1550 ng, about 1575 ng, about 1600 ng, about 1625 ng, about 1650 ng, about 1675 ng, about 1700 ng, about 1725 ng, about 1750 ng, about 1775 ng, about 1800 ng, about 1825 ng, about 1850 ng, about 1875 ng, about 1900 ng, about 1925 ng, about 1950 ng, about 1975 ng, or about 2000 ng) of an active agent.

In some embodiments, the lithe liposome composition comprises about 1 ng to about 800 ng (e.g., about 1 ng, about 2 ng, about 3 ng, about 4 ng, about 5 ng, about 6 ng, about 7 ng, about 8 ng, about 9 ng, about 10 ng, about 20 ng, about 30 ng, about 40 ng, about 50 ng, about 60 ng, about 70 ng, about 80 ng, about 90 ng, about 100 ng, about 110 ng, about 120 ng, about 130 ng, about 140 ng, about 150 ng, about 160 ng, about 170 ng, about 180 ng, about 190 ng, about 200 ng, about 210 ng, about 220 ng, about 230 ng, about 240 ng, about 250 ng, about 260 ng, about 270 ng, about 280 ng, about 290 ng, about 300 ng, about 310 ng, about 320 ng, about 330 ng, about 340 ng, about 350 ng, about 360 ng, about 370 ng, about 380 ng, about 390 ng, about 400 ng, about 410 ng, about 420 ng, about 430 ng, about 440 ng, about 450 ng, about 460 ng, about 470 ng, about 480 ng, about 490 ng, about 500 ng, about 510 ng, about 520 ng, about 530 ng, about 540 ng, about 550 ng, about 560 ng, about 570 ng, about 580 ng, about 590 ng, about 600 ng, about 610 ng, about 620 ng, about 630 ng, about 640 ng, about 650 ng, about 660 ng, about 670 ng, about 680 ng, about 690 ng, about 700 ng, about 710 ng, about 720 ng, about 730 ng, about 740 ng, about 750 ng, about 760 ng, about 770 ng, about 780 ng, about 790 ng, or about 800 ng) of an active agent.

In some embodiments, the lithe liposome composition comprises about 1 mg to about 10 mg (e.g., about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, or about 10 mg) of an active agent.

In some embodiments, the lithe liposome composition is configured to release about 1 ng to about 800 ng (e.g., about 1 ng, about 2 ng, about 3 ng, about 4 ng, about 5 ng, about 6 ng, about 7 ng, about 8 ng, about 9 ng, about 10 ng, about 20 ng, about 30 ng, about 40 ng, about 50 ng, about 60 ng, about 70 ng, about 80 ng, about 90 ng, about 100 ng, about 110 ng, about 120 ng, about 130 ng, about 140 ng, about 150 ng, about 160 ng, about 170 ng, about 180 ng, about 190 ng, about 200 ng, about 210 ng, about 220 ng, about 230 ng, about 240 ng, about 250 ng, about 260 ng, about 270 ng, about 280 ng, about 290 ng, about 300 ng, about 310 ng, about 320 ng, about 330 ng, about 340 ng, about 350 ng, about 360 ng, about 370 ng, about 380 ng, about 390 ng, about 400 ng, about 410 ng, about 420 ng, about 430 ng, about 440 ng, about 450 ng, about 460 ng, about 470 ng, about 480 ng, about 490 ng, about 500 ng, about 510 ng, about 520 ng, about 530 ng, about 540 ng, about 550 ng, about 560 ng, about 570 ng, about 580 ng, about 590 ng, about 600 ng, about 610 ng, about 620 ng, about 630 ng, about 640 ng, about 650 ng, about 660 ng, about 670 ng, about 680 ng, about 690 ng, about 700 ng, about 710 ng, about 720 ng, about 730 ng, about 740 ng, about 750 ng, about 760 ng, about 770 ng, about 780 ng, about 790 ng, or about 800 ng) of an active agent over a predetermined period of time.

As used herein, the term “predetermined period of time” refers to at least about 1, 2, 3, 4, 5, 7, 8, 9, 10, 11, 12, 13, 14, 15, 16, 17, 18, 19, 20, 21, 22, 23, 24, 25, 26, 27, or 28 days or more, or at least 1, 2, 3, 4, 5, 6, 8, 9, 10, 11 or 12 weeks or more, or about 1-10, 10-25, 15-20, 10-20 or 15-25 days.

### (i) Cannabinoids

In some embodiments, the active agent comprises a cannabinoid or a cannabinoid derivative. In some embodiments, the lithe liposome comprises at least one cannabinoid selected from the group consisting of cannabidiol (CBD), cannabidiolic acid (CBDA), cannabinol (CBN), cannabinolic acid (CBNA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabivarinic acid (CBVA), tetrahydrocannabivarin (THCV), tetrahydrocannabivarinic acid (THCVA) cannabidivarin (CBDV), cannabidivarinic acid (CBDVA), cannabichromevarin (CBCV), cannabichromevarinic acid (CBCVA), cannabigerovarin (CBGV), cannabigerovarinic acid (CBGVA), salts thereof, derivatives thereof, and combinations thereof. In some embodiments, the active agent comprises cannabidiol (CBD) or a CBD derivative.

### (ii) Vitamins

In some embodiments, the active agent comprises a vitamin or a vitamin derivative. In some embodiments, the active agent comprises a fat-soluble vitamin or a derivative thereof. In some embodiments, “fat-soluble vitamin” refers to vitamins that may be insoluble in water but may be soluble in fats and nonpolar organic solvents. In some embodiments, “fat soluble vitamin” encompasses vitamin A, vitamin D, vitamin E, vitamin K, and derivatives thereof.

In some embodiments, the lithe liposome comprises at least one vitamin selected from the group consisting of vitamin A, vitamin C, vitamin D2 (ergocalciferol), vitamin D3 (cholecalciferol), vitamin E (α-tocopherol), vitamin K, vitamin K1, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folate), vitamin B12 (cyanocobalamin), salts thereof, derivatives thereof, and combinations thereof. In some embodiments, the active agent comprises Vitamin D3.

In some embodiments, the lithe liposome comprises a quinone or a quinone derivative. In some embodiments, the lithe liposome comprises a Coenzyme Q10 or a Coenzyme Q10 derivative. Coenzyme Q10 (ubiquinone/ubiquinol) is a fat-soluble quinone with a structure similar to that of vitamin K. Coenzyme Q10 is also known as Coenzyme Q, CoQ, CoQ10, Ubiquinone, Ubiquinone-Q10, Ubidecarenone, or Vitamin Q10.

(iii) Plant Alkaloids

In some embodiments, the active agent comprises a plant alkaloid or a derivative of a plant alkaloid.

In some embodiments, the active agent comprises a benzylisoquinoline alkaloid or a derivative of a benzylisoquinoline alkaloid. In some embodiments, the active agent comprises a berberine or a berberine derivative. In some embodiments, the active agent comprises a salt of a berberine, such as a hydrochloride salt form of berberine (also referred to as “berberine hydrochloride” or “berberine HCl”).

### (iv) Curcuminoids

In some embodiments, the active agent comprises a curcuminoid or a derivative of a curcuminoid. Exemplary curcuminoids include, without limitation, curcumin (also referred to as “diferuloylmethane”), demethoxycurcumin (DMC), and bis-demethoxycurcumin (BDMC). In some embodiments, the term “curcuminoid” refers to a mixture of curcumin, demethoxycurcumin, and bisdidemethoxycurcumin. Curcuminoids are active ingredients of the dietary spice Turmeric (Curcuma longa Linn) and may be extracted from the rhizomes of C. longa, a plant in the Zingiberaceae family. In some embodiments, the active agent comprises turmeric and/or an extract of turmeric.

### Exemplary Lithe Liposome Compositions

In some embodiments, the lithe liposome composition may comprise any one of the exemplary lithe liposome composition, Examples 1-11, as set forth in the tables below. One skilled in the art would appreciate that, in some embodiments, the active agent set forth in any one of Examples 1-11 may be exchanged with another active agent, e.g., as described herein. In some embodiments, the active agent set forth in any one of Examples 1-11 may be exchanged with another active agent, e.g., folic acid (folate, vitamin B9), vitamin B12, and/or CoQ10. One skilled in the art would also appreciate that, in some embodiments, the amount of active agent present in any one of Examples 1-11 may vary by, for example, ±20%.

### Example 1

| Components | Content (% W/V) |
|---|---|
| Phosphatidylcholine | 1.9 |
| Cannabidiol | 0.2 |
| Cholesterol | 0.1 |
| Span 60 | 0.5 |
| Hydrophilic matrix | quantum satis (QS) |

### Example 2

| Components | Content (% W/V) |
|---|---|
| Phosphatidylcholine | 2.0 |
| Cannabidiol | 0.2 |
| Cholesterol | 0.1 |
| Polysorbate 80 | 0.5 |
| Quercetin | 0.2 |
| Hydrophilic matrix | QS |

### Example 3

| Components | Content (% W/V) |
|---|---|
| Phosphatidylcholine | 2.1 |
| Berberine HCl | 0.1 |
| Bile Salts | 0.3 |
| Hydrophilic matrix | QS |

### Example 4

| Components | Content (% W/V) |
|---|---|
| Phosphatidylcholine | 0.16 |
| Vitamin D3 | 0.08 |
| Cholesterol | 0.02 |
| Sodium Cholate | 0.04 |
| Hydrophilic matrix | QS |

### Example 5

| Components | Content (% W/V) |
|---|---|
| Phosphatidylcholine | 0.16 |
| Vitamin D3 | 0.08 |
| Cholesterol | 0.02 |
| Dihydroquercetin | 0.05 |
| Sodium Cholate | 0.04 |
| Hydrophilic matrix | QS |

### Example 6

| Components | Content (% W/V) |
|---|---|
| Phosphatidylcholine | 0.16 |
| Vitamin D3 | 0.08 |
| Cholesterol | 0.02 |
| Menthol | 0.02 |
| Sodium Cholate | 0.04 |
| Hydrophilic matrix | QS |

### Example 7

| Components | Content (% W/V) |
|---|---|
| Phosphatidylcholine | 0.06 |
| Vitamin D3 | 0.03 |
| Cholesterol | 0.01 |
| Sodium Cholate | 0.01 |
| Hydrophilic matrix | QS |

### Example 8

| Components | Content (% W/V) |
|---|---|
| Phosphatidylcholine | 1.9 |
| Cannabidiol | 0.2 |
| Cholesterol | 0.2 |
| Span 80 | 0.7 |
| Dihydroquercetin | 0.2 |
| L-Menthol | 0.2 |
| Hydrophilic matrix | QS |

### Example 9

| Components | Content (% W/V) |
|---|---|
| Phosphatidylcholine | 3.0 |
| Vitamin K1 | 2.25 |
| Bile Salts | 1.5 |
| Quercetin | 0.125 |
| Hydrophilic matrix | QS |

### Example 10

| Components | Content (% W/V) |
|---|---|
| Phosphatidylcholine | 2.0 |
| Curcuminoids | 0.25 |
| Hydroxypropyl-β-cyclodextrin | 1.0 |
| Hydrophilic matrix | QS |

### Example 11

| Components | Content (% W/V) |
|---|---|
| Phosphatidylcholine | 12.5 |
| Curcuminoids | 2.0 |
| Bile Salts | 10.0 |
| Quercetin | 0.5 |
| Hydrophilic matrix | QS |

In some embodiments, the lithe liposome composition comprises one or more selected from the group consisting of a phosphatidylcholine, a cannabidiol, a cholesterol, a span 60, a polysorbate 80, a quercetin, a berberine (e.g., berberine HCl), a bile acid, a bile salt, a vitamin D3, a vitamin K1, a curcuminoid, a sodium cholate, a dihydroquercetin, a span 80, menthol (e.g., L-menthol), a hydroxypropyl-β-cyclodextrin (HBC), an hydrophilic matrix, any salt thereof, any derivative thereof, and any combination thereof.

In some embodiments, the lithe liposome composition comprises about 0.01% w/v to about 50% w/v (e.g., about 0.01% w/v, about 0.05% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, about 9.5% w/v, about 10% w/v, about 15% w/v, about 20% w/v, about 25% w/v, about 30% w/v, about 35% w/v, about 40% w/v, about 45% w/v, or about 50% w/v) of one or more selected from the group consisting of a phosphatidylcholine, a cannabidiol, a cholesterol, a span 60, a polysorbate 80, a quercetin, a berberine (e.g., berberine HCl), a bile acid, a bile salt, a vitamin D3, a vitamin K1, a curcuminoid, a sodium cholate, a dihydroquercetin, a span 80, menthol (e.g., L-menthol), a hydroxypropyl-β-cyclodextrin (HBC), an hydrophilic matrix, any salt thereof, any derivative thereof, and any combination thereof.

In some embodiments, the lithe liposome composition comprises about 0.01% w/v to about 1% w/v (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.11% w/v, about 0.12% w/v, about 0.13% w/v, about 0.14% w/v, about 0.15% w/v, about 0.16% w/v, about 0.17% w/v, about 0.18% w/v, about 0.19% w/v, about 0.2% w/v, about 0.21% w/v, about 0.22% w/v, about 0.23% w/v, about 0.24% w/v, about 0.25% w/v, about 0.26% w/v, about 0.27% w/v, about 0.28% w/v, about 0.29% w/v, about 0.3% w/v, about 0.31% w/v, about 0.32% w/v, about 0.33% w/v, about 0.34% w/v, about 0.35% w/v, about 0.36% w/v, about 0.37% w/v, about 0.38% w/v, about 0.39% w/v, about 0.4% w/v, about 0.41% w/v, about 0.42% w/v, about 0.43% w/v, about 0.44% w/v, about 0.45% w/v, about 0.46% w/v, about 0.47% w/v, about 0.48% w/v, about 0.49% w/v, about 0.5% w/v, about 0.51% w/v, about 0.52% w/v, about 0.53% w/v, about 0.54% w/v, about 0.55% w/v, about 0.56% w/v, about 0.57% w/v, about 0.58% w/v, about 0.59% w/v, about 0.6% w/v, about 0.61% w/v, about 0.62% w/v, about 0.63% w/v, about 0.64% w/v, about 0.65% w/v, about 0.66% w/v, about 0.67% w/v, about 0.68% w/v, about 0.69% w/v, about 0.7% w/v, about 0.71% w/v, about 0.72% w/v, about 0.73% w/v, about 0.74% w/v, about 0.75% w/v, about 0.76% w/v, about 0.77% w/v, about 0.78% w/v, about 0.79% w/v, about 0.8% w/v, about 0.81% w/v, about 0.82% w/v, about 0.83% w/v, about 0.84% w/v, about 0.85% w/v, about 0.86% w/v, about 0.87% w/v, about 0.88% w/v, about 0.89% w/v, about 0.9% w/v, about 0.91% w/v, about 0.92% w/v, about 0.93% w/v, about 0.94% w/v, about 0.95% w/v, about 0.96% w/v, about 0.97% w/v, about 0.98% w/v, about 0.99% w/v, or about 1% w/v) of one or more selected from the group consisting of a phosphatidylcholine, a cannabidiol, a cholesterol, a span 60, a polysorbate 80, a quercetin, a berberine (e.g., berberine HCl), a bile acid, a bile salt, a vitamin D3, a vitamin K1, a curcuminoid, a sodium cholate, a dihydroquercetin, a span 80, menthol (e.g., L-menthol), a hydroxypropyl-β-cyclodextrin (HBC), an hydrophilic matrix, any salt thereof, any derivative thereof, and any combination thereof.

In some embodiments, the lithe liposome composition comprises about 1% w/v to about 10% w/v (e.g., about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, about 5.1% w/v, about 5.2% w/v, about 5.3% w/v, about 5.4% w/v, about 5.5% w/v, about 5.6% w/v, about 5.7% w/v, about 5.8% w/v, about 5.9% w/v, about 6% w/v, about 6.1% w/v, about 6.2% w/v, about 6.3% w/v, about 6.4% w/v, about 6.5% w/v, about 6.6% w/v, about 6.7% w/v, about 6.8% w/v, about 6.9% w/v, about 7% w/v, about 7.1% w/v, about 7.2% w/v, about 7.3% w/v, about 7.4% w/v, about 7.5% w/v, about 7.6% w/v, about 7.7% w/v, about 7.8% w/v, about 7.9% w/v, about 8% w/v, about 8.1% w/v, about 8.2% w/v, about 8.3% w/v, about 8.4% w/v, about 8.5% w/v, about 8.6% w/v, about 8.7% w/v, about 8.8% w/v, about 8.9% w/v, about 9% w/v, about 9.1% w/v, about 9.2% w/v, about 9.3% w/v, about 9.4% w/v, about 9.5% w/v, about 9.6% w/v, about 9.7% w/v, about 9.8% w/v, about 9.9% w/v, or about 10% w/v) of one or more selected from the group consisting of a phosphatidylcholine, a cannabidiol, a cholesterol, a span 60, a polysorbate 80, a quercetin, a berberine (e.g., berberine HCl), a bile acid, a bile salt, a vitamin D3, a vitamin K1, a curcuminoid, a sodium cholate, a dihydroquercetin, a span 80, menthol (e.g., L-menthol), a hydroxypropyl-β-cyclodextrin (HBC), an hydrophilic matrix, any salt thereof, any derivative thereof, and any combination thereof.

In some embodiments, the composition comprises (i) a phospholipid, an active agent, a cholesterol or a derivative thereof, a lipid bilayer modifier and/or a hydrophilic matrix; (ii) a phospholipid, an active agent, a cholesterol or a derivative thereof, a lipid bilayer modifier, an absorption enhancer, and/or a hydrophilic matrix; (iii) a phospholipid, an active agent, a cholesterol or a derivative thereof, a lipid bilayer modifier, optionally comprising a surfactant, an absorption enhancer, an additional lipid bilayer modifier, optionally comprising a solvent, and/or a hydrophilic matrix. (iv) a phospholipid, an active agent, a lipid bilayer modifier, and/or a hydrophilic matrix; or (v) a phospholipid, an active agent, a lipid bilayer modifier, an absorption enhancer, and/or a hydrophilic matrix.

In some embodiments, the composition comprises a phospholipid comprising a phosphatidylcholine (PC) or a derivative thereof; an active agent comprising a cannabidiol (CBD) or a derivative thereof; a cholesterol or a derivative thereof; a lipid bilayer modifier comprising span 60 or a derivative thereof; and/or a hydrophilic matrix.

In some embodiments, the composition comprises a phospholipid comprising a phosphatidylcholine (PC) or a derivative thereof; an active agent comprising a cannabidiol (CBD) or a derivative thereof; a cholesterol or a derivative thereof; a lipid bilayer modifier comprising a polysorbate 80 or a derivative thereof; an absorption enhancer comprising a quercetin or a derivative thereof; and/or a hydrophilic matrix.

In some embodiments, the composition comprises a phospholipid comprising a phosphatidylcholine (PC) or a derivative thereof; an active agent comprising a berberine hydrochloride or a derivative thereof; a lipid bilayer modifier comprising a bile acid, a bile salt, or a derivative thereof; and/or a hydrophilic matrix.

In some embodiments, the composition comprises a phospholipid comprising a phosphatidylcholine (PC) or a derivative thereof; an active agent comprising a Vitamin D3 or a derivative thereof; a cholesterol or a derivative thereof; a lipid bilayer modifier comprising a sodium cholate or a derivative thereof; and/or a hydrophilic matrix.

In some embodiments, the composition comprises a phospholipid comprising a phosphatidylcholine (PC) or a derivative thereof; an active agent comprising a Vitamin D3 or a derivative thereof; a cholesterol or a derivative thereof; an absorption enhancer comprising a dihydroquercetin or a derivative thereof; a lipid bilayer modifier comprising sodium cholate; and/or a hydrophilic matrix.

In some embodiments, the composition comprises a phospholipid comprising a phosphatidylcholine (PC) or a derivative thereof; an active agent comprising a Vitamin D3 or a derivative thereof; a cholesterol or a derivative thereof; an absorption enhancer comprising menthol; a lipid bilayer modifier comprising sodium cholate or a derivative thereof; and/or a hydrophilic matrix. 31. The composition of any one of the preceding claims, comprising: a phospholipid comprising a phosphatidylcholine (PC) or a derivative thereof; an active agent comprising a Vitamin D3 or a derivative thereof; a cholesterol or a derivative thereof; a lipid bilayer modifier comprising a sodium cholate or a derivative thereof; and/or a hydrophilic matrix.

In some embodiments, the composition comprises a phospholipid comprising a phosphatidylcholine (PC) or a derivative thereof; an active agent comprising a cannabidiol (CBD) or a derivative thereof; a cholesterol or a derivative thereof; a lipid bilayer modifier comprising a surfactant, optionally wherein the surfactant comprises span 80 or a derivative thereof; an absorption enhancer comprising a dihydroquercetin or a derivative thereof; a lipid bilayer modifier comprising a solvent, optionally wherein the solvent comprises methanol or a derivative thereof; and/or a hydrophilic matrix.

In some embodiments, the composition comprises a phospholipid comprising a phosphatidylcholine (PC) or a derivative thereof; an active agent comprising a Vitamin K1 or a derivative thereof; a lipid bilayer modifier comprising a bile acid, a bile salt, or a derivative thereof; an absorption enhancer comprising a quercetin or a derivative thereof; and/or a hydrophilic matrix.

In some embodiments, the composition comprises a phospholipid comprising a phosphatidylcholine (PC) or a derivative thereof; an active agent comprising a curcuminoid or a derivative thereof; a lipid bilayer modifier comprising a hydroxypropyl-β-cyclodextrin (HBC) or a derivative thereof; and/or a hydrophilic matrix.

In some embodiments, the composition comprises a phospholipid comprising a phosphatidylcholine (PC) or a derivative thereof; an active agent comprising a curcuminoid or a derivative thereof; a lipid bilayer modifier comprising a bile acid, a bile salt, or a derivative thereof; an absorption enhancer comprising a quercetin or a derivative thereof; and/or a hydrophilic matrix.

In some embodiments, the lithe liposome composition comprises a phospholipid, optionally a phosphatidylcholine (PC) or a derivative thereof, in an amount of about 1.9% w/v; an active agent, optionally a cannabidiol (CBD) or a derivative thereof, in an amount of about 0.2% w/v; a cholesterol or a derivative thereof in an amount of about 0.1% w/v; a lipid bilayer modifier, optionally span 60 or a derivative thereof, in an amount of about 0.5% w/v; and/or quantum satis (QS) a hydrophilic matrix.

In some embodiments, the lithe liposome composition comprises a phospholipid, optionally a phosphatidylcholine (PC) or a derivative thereof, in an amount of about 2.0% w/v; an active agent, optionally a cannabidiol (CBD) or a derivative thereof, in an amount of about 0.2% w/v; a cholesterol or a derivative thereof in an amount of about 0.1% w/v; a lipid bilayer modifier, optionally a polysorbate 80 or a derivative thereof, in an amount of about 0.5% w/v; an absorption enhancer, optionally a quercetin or a derivative thereof, in an amount of about 0.2% w/v; and/or quantum satis (QS) a hydrophilic matrix.

In some embodiments, the lithe liposome composition comprises a phospholipid, optionally a phosphatidylcholine (PC) or a derivative thereof, in an amount of about 2.1% w/v; an active agent, optionally a berberine hydrochloride or a derivative thereof, in an amount of about 0.1% w/v; a lipid bilayer modifier, optionally a bile acid, a bile salt, or a derivative thereof, in an amount of about 0.3% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.16% w/v; an active agent, optionally a Vitamin D3 or a derivative thereof, in an amount of about 0.08% w/v; a cholesterol or a derivative thereof in an amount of about 0.02% w/v; a lipid bilayer modifier, optionally a sodium cholate or a derivative thereof, in an amount of about 0.04% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.16% w/v; an active agent, optionally a Vitamin D3, in an amount of about 0.08% w/v; a cholesterol or a derivative thereof in an amount of about 0.02% w/v; an absorption enhancer, optionally a dihydroquercetin or a derivative thereof, in an amount of about 0.05% w/v; a lipid bilayer modifier, optionally sodium cholate, in an amount of about 0.04% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.16% w/v; an active agent, optionally a Vitamin D3, in an amount of about 0.08% w/v; a cholesterol or a derivative thereof in an amount of about 0.02% w/v; an absorption enhancer, optionally menthol, in an amount of about 0.02% w/v; a lipid bilayer modifier, optionally sodium cholate or a derivative thereof, in an amount of about 0.04% w/v; and/or quantum satis (QS) a hydrophilic matrix.

In some embodiments, the lithe liposome composition comprises a phospholipid, optionally a phosphatidylcholine (PC) or a derivative thereof, in an amount of about 0.06% w/v; an active agent, optionally a Vitamin D3 or a derivative thereof, in an amount of about 0.03% w/v; a cholesterol or a derivative thereof in an amount of about 0.01% w/v; a lipid bilayer modifier, optionally a sodium cholate or a derivative thereof, in an amount of about 0.01% w/v; and/or quantum satis (QS) a hydrophilic matrix.

In some embodiments, the lithe liposome composition comprises a phospholipid, optionally a phosphatidylcholine (PC) or a derivative thereof, in an amount of about 1.9% w/v; an active agent, optionally a cannabidiol (CBD) or a derivative thereof, in an amount of about 0.2% w/v; a cholesterol or a derivative thereof in an amount of about 0.2% w/v; a lipid bilayer modifier comprising a surfactant, optionally span 80 or a derivative thereof, in an amount of about 0.7% w/v; an absorption enhancer, optionally a dihydroquercetin or a derivative thereof, in an amount of about 0.2% w/v; a lipid bilayer modifier comprising a solvent, optionally methanol or a derivative thereof, in an amount of about 0.2% w/v; and/or quantum satis (QS) a hydrophilic matrix.

In some embodiments, the lithe liposome composition comprises a phospholipid, optionally a phosphatidylcholine (PC) or a derivative thereof, in an amount of about 3.0% w/v; an active agent, optionally a Vitamin K1 or a derivative thereof, in an amount of about 2.25% w/v; a lipid bilayer modifier, optionally a bile acid, a bile salt, or a derivative thereof, in an amount of about 1.5% w/v; an absorption enhancer, optionally a quercetin or a derivative thereof, in an amount of about 0.125% w/v; and/or quantum satis (QS) a hydrophilic matrix.

In some embodiments, the lithe liposome composition comprises a phospholipid, optionally a phosphatidylcholine (PC) or a derivative thereof, in an amount of about 2.0% w/v; an active agent, optionally a curcuminoid or a derivative thereof, in an amount of about 0.25% w/v; a lipid bilayer modifier, optionally a hydroxypropyl-β-cyclodextrin (HBC) or a derivative thereof, in an amount of about 1.0% w/v; and/or quantum satis (QS) a hydrophilic matrix.

In some embodiments, the lithe liposome composition comprises a phospholipid, optionally a phosphatidylcholine (PC) or a derivative thereof, in an amount of about 12.5% w/v; an active agent, optionally a curcuminoid or a derivative thereof, in an amount of about 2% w/v; a lipid bilayer modifier, optionally a bile acid, a bile salt, or a derivative thereof, in an amount of about 10% w/v; an absorption enhancer, optionally a quercetin or a derivative thereof, in an amount of about 0.5% w/v; and/or quantum satis (QS) a hydrophilic matrix.

In some embodiments, the lithe liposome composition comprises quantum satis (QS) a hydrophilic matrix, such as a hydrophilic solution. In some embodiments, the lithe liposome composition comprises quantum satis (QS) a hydrophilic matrix to about 0% w/v to about 100% w/v (e.g., about 1% w/v, about 5% w/v, about 10% w/v, about 15% w/v, about 20% w/v, about 25% w/v, about 30% w/v, about 35% w/v, about 40% w/v, about 45% w/v, about 50% w/v, about 55% w/v, about 60% w/v, about 65% w/v, about 70% w/v, about 75% w/v, about 80% w/v, about 85% w/v, about 90% w/v, about 95% w/v, or about 100% w/v).

In some embodiments, the lithe liposome composition comprises about 5 mL to about 5000 L of a hydrophilic matrix.

### Methods of Making

The present disclosure provides methods of making lithe liposome compositions.

In some embodiments, the lithe liposome compositions of the present disclosure may be prepared by a method, comprising: (i) dissolving a phospholipid in an organic solvent to form a lipid mixture; (ii) optionally contacting the lipid mixture with a lipid bilayer modifier, an absorption enhancer, or a combination thereof; (iii) contacting the lipid mixture with an active agent, such as one or more active agent(s) described herein; (iv) drying the lipid mixture to remove the organic solvent to form a dried lipid thin film; (v) hydrating the dried lipid thin film with a hydrophilic solution (e.g., an aqueous solution) to form a liposomal suspension; (vi) optionally contacting the liposomal suspension with a lipid bilayer modifier, an absorption enhancer, or a combination thereof; (vii) optionally contacting the liposomal suspension with one or more hydrophilic active agent(s); and (viii) optionally agitating, stirring, and/or sonicating the liposomal suspension. In some embodiments, the lipid bilayer modifier, the absorption enhancer, or the combination thereof can be added before drying the lipid mixture, after drying the lipid mixture, or both before and after drying the lipid mixture. In some embodiments, the lipid bilayer modifier, the absorption enhancer, or the combination thereof can be added before hydrating the dried lipid thin film, after hydrating the dried lipid thin film, or both before and after hydrating the dried lipid thin film.

In some embodiments, the lithe liposome compositions of the present disclosure may be prepared by a method, comprising encapsulating an active agent into a lithe liposome composition (e.g., by dissolving a phospholipid in an organic solvent to form a lipid mixture, and contacting the lipid mixture with an active agent). In some embodiments, the method comprises contacting a lithe liposome composition of the present disclosure with an active agent. In one embodiment, the lithe liposome composition is contacted with an active agent in an aqueous medium.

In certain embodiments, the lithe liposome compositions of the present disclosure may be prepared using the thin-film hydration technique. In some embodiments, the phospholipid ingredients are dissolved in an organic solvent, such as dichloromethane, chloroform, ethanol, or a chloroform-methanol mixture. In some embodiments, drying the lipid mixture to remove the organic solvent comprises evaporation under vacuum pump at a temperature of 40-70° C. In some embodiments, for small volumes (e.g., <1 mL), the organic solvent may be evaporated by means of a dry nitrogen or argon stream in a fume hood until the residual organic solvent is completely removed. In some embodiments, for larger volumes (e.g., >1 mL) a rotary evaporation may be used to remove organic solvent. In some embodiments, the lipid mixture can be evaporated under a nitrogen gas stream and the obtained film can optionally be placed in a vacuum desiccator overnight to remove any remaining solvent. In some embodiments, after the removal of the organic solvent, a homogeneous, dry, thin-lipid film (of stacked bilayers) is formed. In some embodiments, the lipid film may be hydrated using an appropriate aqueous medium (e.g., buffer) that, for the pharmaceutical formulation, may comprise a solution of simple distilled water, a normal (e.g., phosphate) saline buffer or other buffer solution. In some embodiments, the dried lipid film may be hydrated with phosphate saline buffer and sonicated in a sonicator bath for 1 hour followed by 15 minutes of probe sonication in an ice-water bath. In some embodiments, the liposomal suspension may be centrifuged for 30 minutes at 3,000 g. In some embodiments, the hydration process (with a duration of about 1 hour to about 2 hour) may be performed at a temperature of 60-70° C., or at a temperature above the phase-transition temperature of the component lipids. In some embodiments, during the hydration stage, agitation, stirring, and/or sonication may be helpful to detach the (swelling) lipids' lamellae from the internal vessel surface. In some embodiments, to facilitate full lipid hydration, the final liposome suspension may be left overnight at a temperature of about 4° C.

### Methods of Use

The present disclosure provides methods of using lithe liposome compositions.

In some embodiments, the present invention provides a method of delivering an active agent to a subject in need thereof. The method comprises administering to the subject a lithe liposome composition comprising the active agent, as described herein.

In some embodiments, the present invention provides a method of enhancing the stability of an active agent in the gastrointestinal tract of a subject. The method comprises administering to the subject a lithe liposome composition of the present disclosure comprising one or more active agents. In some embodiments, the administration comprising oral administration.

In some embodiments, the present invention provides a method of improving the permeability of an active agent across the intestinal epithelia of a subject. The method comprises administering to the subject a lithe liposome composition of the present disclosure comprising the active agent. In some embodiments, the administration comprising oral administration.

In some embodiments, the present invention provides a method of improving the oral absorption, the oral bioavailability, and/or the bio-variability of an active agent in a subject. The method comprises administering to the subject a lithe liposome composition of the present disclosure comprising the active agent. In some embodiments, the administration comprising oral administration.

In some embodiments, the present invention provides a method of treating and/or preventing a disease or condition in a subject. The method comprises administering to the subject a lithe liposome composition of the present disclosure comprising an active agent. In some embodiments, the administration comprising oral administration.

### Pharmaceutical and Nutraceutical Compositions

The present disclosure encompasses the preparation and use of pharmaceutical compositions and/or nutraceutical compositions comprising a lithe liposome of the disclosure. Such a pharmaceutical composition and/or nutraceutical composition may consist of the lithe liposome alone, as a combination of at least one lithe liposome (e.g., an effective dose of a lithe liposome) in a form suitable for administration to a subject, or the pharmaceutical composition and/or nutraceutical composition may comprise the lithe liposome and one or more pharmaceutically acceptable carriers, one or more additional (active and/or inactive) ingredients, or some combination thereof.

The pharmaceutical compositions and/or nutraceutical compositions of the disclosure may contain a therapeutically effective amount of a lithe liposome described herein. Those skilled in the art will recognize, however, that a pharmaceutical composition and/or nutraceutical composition may contain more than a therapeutically effective amount, such as in bulk compositions, or less than a therapeutically effective amount, that is, individual unit doses designed for multiple administration to achieve a therapeutically effective amount. Typically, the actual amount will depend upon the formulation itself, the route of administration, the frequency of dosing, and so forth.

According to some aspects, the lithe liposome of the disclosure, may be incorporated into pharmaceutical compositions suitable for administration, e.g., oral administration.

According to some aspects, the lithe liposome of the disclosure, may be incorporated into nutraceutical compositions suitable for administration, e.g., oral administration.

Pharmaceutical compositions and/or nutraceutical compositions of the present disclosure may be administered in a manner appropriate to the condition, disease, and/or disorder to be treated (or prevented). The quantity and frequency of administration will be determined by such factors as the condition of the patient, and the type and severity of the patient's condition, disease, and/or disorder, although appropriate dosages may be determined by clinical trials.

The administration of the pharmaceutical compositions and/or nutraceutical compositions may be carried out in any convenient manner and by any route of administration including, but not limited to, oral, parenteral, intraperitoneal, intravenous, intraarterial, buccal, nasal, aerosol, sublingual, intramuscular, rectal, transbuccal, intranasal, vaginal, intraocular, subcutaneous, intraadipose, intraarticular, intracerebroventricular, intraperitoneal, intrathecal, inhalation, injection, ingestion, transfusion, implantation, or transplantation.

As used herein, the term “pharmaceutically acceptable carrier” means a chemical composition with which the active agent may be combined and which, following the combination, can be used to administer the active agent to a subject. Suitable carriers are described in the most recent edition of Remington's Pharmaceutical Sciences, a standard reference text in the field, which is incorporated herein by reference.

The formulations of the pharmaceutical compositions and/or nutraceutical compositions described herein may be prepared by any method known or hereafter developed in the art of pharmacology. In general, such preparatory methods include the step of bringing the lithe liposome into association with a carrier or one or more other accessory ingredients, and then, if necessary or desirable, shaping or packaging the product into a desired single- or multi-dose unit.

In certain embodiments, the pharmaceutical compositions and/or nutraceutical compositions of the present disclosure may be a lithe liposomal aqueous suspension. In certain embodiments, the lithe liposomal aqueous suspension can be further incorporated into different dosage forms, including but not limiting to, soft/hard shell capsule, solution, syrup, tablet, sprinkler, inhaler, spray, suspension, paste, emulsion, gel, patch, and/or microneedles.

### EXAMPLES

### Example 1: Lithe Liposome Generation and Characterization

### Generation of Lithe Liposome Compositions

The specific amount of each component of the lithe liposome compositions (Examples 1-8) were first dissolved in an organic solvent or solvent mixture (e.g., ethanol, chloroform, methanol, or the mixture of these, etc.). The solvent was then evaporated under a nitrogen gas stream or by rotary evaporation to generate a dried lipid film. The dried lipid film was subsequently hydrated with phosphate buffer saline (pH 7.4), purified water, or other hydrophilic solvent systems to obtain a liposomal solution. The obtained liposomal solution was sonicated in a sonication bath, probe sonicator, or homogenizer in an ice-water bath to obtain a liposomal suspension. The liposomal suspension was then centrifuged for 30 minutes at 3,000 g. The supernatant was collected in airtight containers and stored under refrigerated conditions prior to use.

As used in Examples 1-8, below, the term “quantum satis” or “QS” refers to the addition of a predetermined amount of a hydrophilic matrix, e.g., phosphate buffer saline (pH 7.4) or other hydrophilic solvent systems, used to obtain the liposomal solution. In some embodiments, the predetermined amount of a hydrophilic matrix may comprise about 5 mL to about 5000 L. In some embodiments, the predetermined amount of a hydrophilic matrix may comprise about 5 mL to about 1000 L.

In some embodiments, when the liposomal solution is produced as a commercial batch, the predetermined amount of a hydrophilic matrix may comprise about 5 mL to about 5000 L. In some embodiments, the commercial batch size may be about 5 mL to about 1000 L. In some embodiments, the commercial batch size may be about 1000 L or more.

### Example 1

| Components | Content (% W/V) |
|---|---|
| Phosphatidylcholine | 1.9 |
| Cannabidiol | 0.2 |
| Cholesterol | 0.1 |
| Span 60 | 0.5 |
| Hydrophilic matrix | quantum satis (QS) |

### Example 2

| Components | Content (% W/V) |
|---|---|
| Phosphatidylcholine | 2.0 |
| Cannabidiol | 0.2 |
| Cholesterol | 0.1 |
| Polysorbate 80 | 0.5 |
| Quercetin | 0.2 |
| Hydrophilic matrix | QS |

### Example 3

| Components | Content (% W/V) |
|---|---|
| Phosphatidylcholine | 2.1 |
| Berberine HCl | 0.1 |
| Bile Salts | 0.3 |
| Hydrophilic matrix | QS |

### Example 4

| Components | Content (% W/V) |
|---|---|
| Phosphatidylcholine | 0.16 |
| Vitamin D3 | 0.08 |
| Cholesterol | 0.02 |
| Sodium Cholate | 0.04 |
| Hydrophilic matrix | QS |

### Example 5

| Components | Content (% W/V) |
|---|---|
| Phosphatidylcholine | 0.16 |
| Vitamin D3 | 0.08 |
| Cholesterol | 0.02 |
| Dihydroquercetin | 0.05 |
| Sodium Cholate | 0.04 |
| Hydrophilic matrix | QS |

### Example 6

| Components | Content (% W/V) |
|---|---|
| Phosphatidylcholine | 0.16 |
| Vitamin D3 | 0.08 |
| Cholesterol | 0.02 |
| Menthol | 0.02 |
| Sodium Cholate | 0.04 |
| Hydrophilic matrix | QS |

### Example 7

| Components | Content (% W/V) |
|---|---|
| Phosphatidylcholine | 0.06 |
| Vitamin D3 | 0.03 |
| Cholesterol | 0.01 |
| Sodium Cholate | 0.01 |
| Hydrophilic matrix | QS |

### Example 8

| Components | Content (% W/V) |
|---|---|
| Phosphatidylcholine | 1.9 |
| Cannabidiol | 0.2 |
| Cholesterol | 0.2 |
| Span 80 | 0.7 |
| Dihydroquercetin | 0.2 |
| L-Menthol | 0.2 |
| Hydrophilic matrix | QS |

### Generation of Oil-Based Reference Compositions

The specified amount of each component for the oil-based reference compositions (Controls 1-2) were weighed and transferred into a suitable container. The weighed components were mixed with the aid of a stirrer mixer or homogenizer at room temperature until a homogeneous mixture was obtained.

### Oil-Based Reference Composition (Reference 1)

| Components | Content (% W/V) |
|---|---|
| Vitamin D3 | 0.08 |
| Medium Chain Triglycerides (MCT) Oil | QS |

Oil-Based Reference Composition with Sunflower Lecithin (Reference 2)

| Components | Content (% W/V) |
|---|---|
| Vitamin D3 | 0.08 |
| Sunflower Lecithin | 73 |
| MCT/Sunflower Oil Mixture | QS |

Comparison of In Vitro Release of Vitamin D3 from Lithe Liposome Compositions and Conventional Drug Delivery Systems

### Comparative Study 1

The in vitro release of active agents from lithe liposome compositions as compared to conventional oil-based delivery systems was characterized using a dialysis method.

Dialysis bags containing active agent(s) in oil-based formulations (Reference 1), in oil-based formulations with sunflower lecithin (Reference 2), and in lithe liposome compositions (Example 4) were maintained in a bath filled with phosphate buffer saline under constant stirring at 100 rpm at 37° C. with a thermostatic control. At appropriate time intervals, aliquot of dialysis solution was collected and assayed spectrophotometrically for active agent contents. Active agent release profiles were graphed and compared among the testing groups.

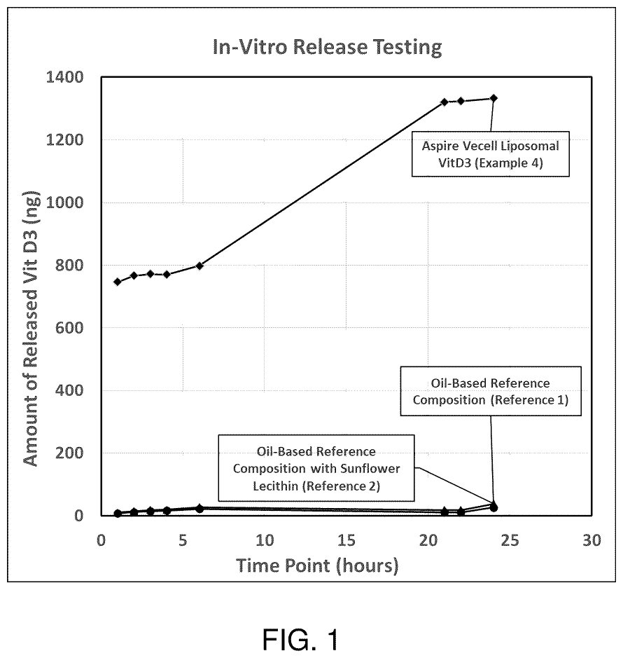

As shown in FIG. 1, in comparison to the oil-based reference formulation and oil-based formulation with Sunflower Lecithin, the lithe liposome composition increased the in vitro release of Vitamin D3.

### Comparative Study 2

The comparative in vitro release of active agents among the variations of lithe liposome compositions was characterized using a dialysis method.

Dialysis bags containing Example 4 (lithe liposome without flavonoid or menthol), Example 5 (lithe liposome with flavonoid) and Example 6 (lithe liposome with menthol) were maintained in a bath filled with phosphate buffer saline under constant stirring at 100 rpm at 37° C. with a thermostatic control. At appropriate time intervals, aliquot of dialysis solution was collected and assayed spectrophotometrically for active agent contents. Active agent release profiles were graphed and compared among the testing groups.

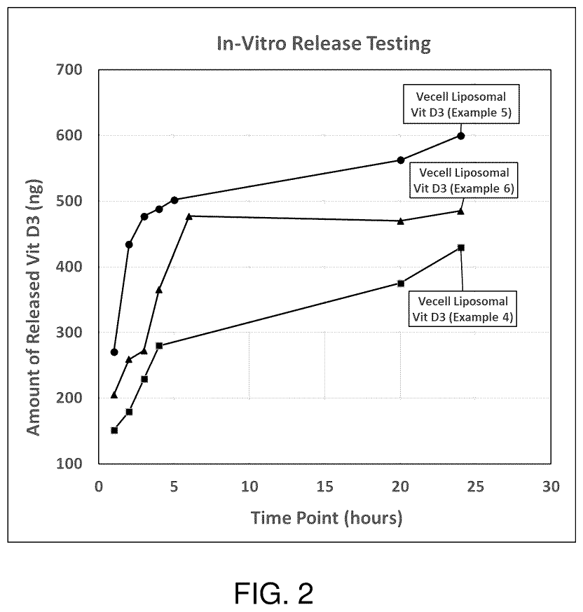

As shown in FIG. 2, lithe liposome compositions containing an absorption enhancer, e.g., flavonoid or menthol, enhanced the in vitro release of Vitamin D3 compared to the lithe liposome composition without an absorption enhancer.

### Example 2: Characterization of VeCell Liposomal Delivery Technology Platform

### Example 9

| Components | Content (% W/V) |
|---|---|
| Phosphatidylcholine | 3.0 |
| Vitamin K1 | 2.25 |
| Bile Salts | 1.5 |
| Quercetin | 0.125 |
| Hydrophilic matrix | quantum satis (QS) |

### Example 10

| Components | Content (% W/V) |
|---|---|
| Phosphatidylcholine | 2.0 |
| Curcuminoids | 0.25 |
| Hydroxypropyl-β-cyclodextrin | 1.0 |
| Hydrophilic matrix | QS |

### Example 11

| Components | Content (% W/V) |
|---|---|
| Phosphatidylcholine | 12.5 |
| Curcuminoids | 2.0 |
| Bile Salts | 10.0 |
| Quercetin | 0.5 |
| Hydrophilic matrix | QS |

### Morphology of Vecell Liposomes

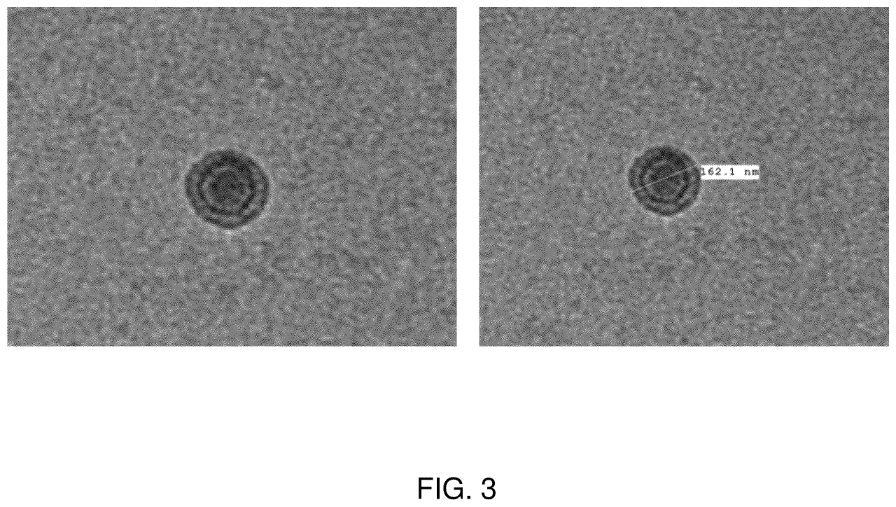

The morphology of Vecell liposomal vesicles was characterized by transmission electron microscopy (TEM). Vecell liposomal vesicles were dispersed in DI-water using a vortex mixer. One drop of liposomal vesicle solution was placed onto a copper grid and the excess solution was immediately adsorbed using filter paper. The sample was then stained by adding a drop of 1% phosphotungstic acid. The excess solution was removed by filter paper, and then the sample was dried at room temperature. Afterward, the grid was observed using a TEM. Vesicle with characteristic lipid bilayer structure was observed and demonstrated in FIG. 3.

### Solubility Test

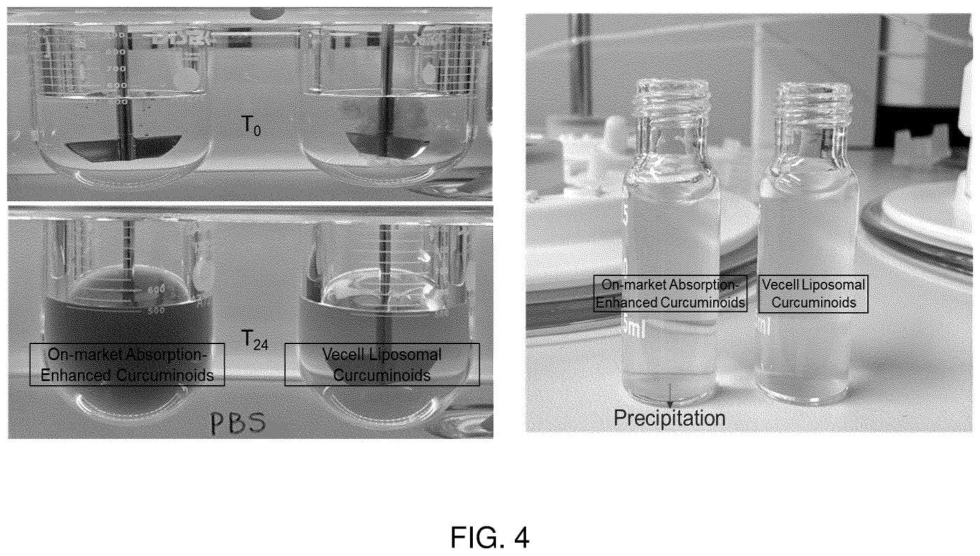

Solubility of active compound in biorelevant media was studied using a USP Dissolution Apparatus II. Four simulated bio-media were selected: PBS (Phosphate-buffered Saline), SGF (Simulated Gastric Fluid), FaSSIF (Fasted State Simulated Intestinal Fluid) and FeSSIF (Fed State Simulated Intestinal Fluid). Methods for preparing PBS, SGF, FaSSIF, and FeSSIF are known in the art. See, e.g., Current USP/NF (US pharmacopeia); and Marques, Margareth. (2004). Dissolution Media Simulating Fasted and Fed States. Dissolution Technologies, the contents of which are incorporated herein by reference in their entirety. On-market Absorption-enhanced Curcuminoids and Vecell Liposomal (Example 11) formulations containing the same amount of curcuminoids were placed in dissolution vessels containing 900 mL medium under constant stirring at 75 rpm and maintained at 37° C. The On-market Absorption-Enhanced product comprises curcuminoids, cyclodextrin, turmeric, magnesium stearate, and silicon dioxide. Samples were collected at 24 hours and assayed spectrophotometrically for the concentration of the dissolved curcuminoids. FIG. 4 shows a picture of the study and indicates that the Vecell Liposomal Curcuminoids is soluble in PBS buffer from 0 to 24 hours, while undissolved particles appear in On-Market Absorption-enhanced product throughout 24 hours. The solubility results are summarized in below Table 2, demonstrating a significant increase of curcuminoids solubility in all four bio-relevant media.

TABLE 2
Solubility (mg/mL)

| Buffer | Reference | Vecell (Example 11) |
|---|---|---|
| PBS | — | 9.68 |
| SGF | 0.002 | 10.01 |
| FaSSIF | 0.001 | 9.96 |
| FeSSIF | 0.015 | 10.15 |

### Permeability Test

The Parallel Artificial Membrane Permeation Assays (PAMPA) is a cell-free assay based on the passive diffusion of the target compound through a lipid-infused artificial membrane to predict its passive, transcellular permeability, e.g., across the intestinal epithelia.

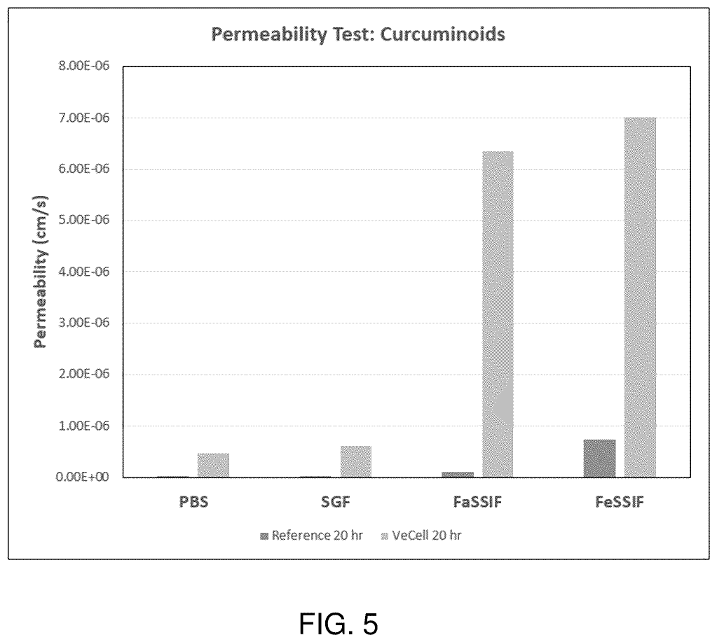

On-market Absorption-enhanced Curcuminoids and Vecell Liposomal (Example 11) formulations containing the same amount of curcuminoids in four bio-relevant media were added to the donor compartment of the PAMPA plate. The acceptors were filled with PBS and the entire content in each acceptor was collected after twenty-hour incubation at 37° C. The permeation of curcuminoids across the artificial phospholipid membrane was quantified spectrophotometrically. The obtained results are graphed in FIG. 5.

As demonstrated in the graph of FIG. 5, permeability of Curcuminoids in Vecell formulation is greatly enhanced by more than 10 times in all 4 bio-relevant buffers. Unlike the reference formulation, the permeability of curcuminoids is more consistent in FaSSIF and FeSSIF, indicating a more comparable absorption under fast and fed conditions.

### Preclinical Animal Study

### Comparative Study 1: VeCell Liposomal Vitamin D3 vs Oil-Based Formulation and Classic Liposomal Formulation

Evaluate the bioavailability of Vecell liposomal Vitamin D3 (Example 5) compared to traditional oil-based Vitamin D3 supplement (Reference 1) and Classic Liposomal Formulation using an animal model, i.e., Sprague Dawley rats. The Classic Liposomal Formulation comprises phosphatidylcholine, vitamin D3, and hydrophilic matrix.

The rats were randomly allocated into 3 groups (n=6 each). Baseline blood samples (TO) were collected one-day prior. On the study day, one dose of the Vitamin D3 formulations was administrated to the animals in the morning. Blood withdrawals were performed at times points 2, 4, 8, 16 and 24 hours after oral administration.

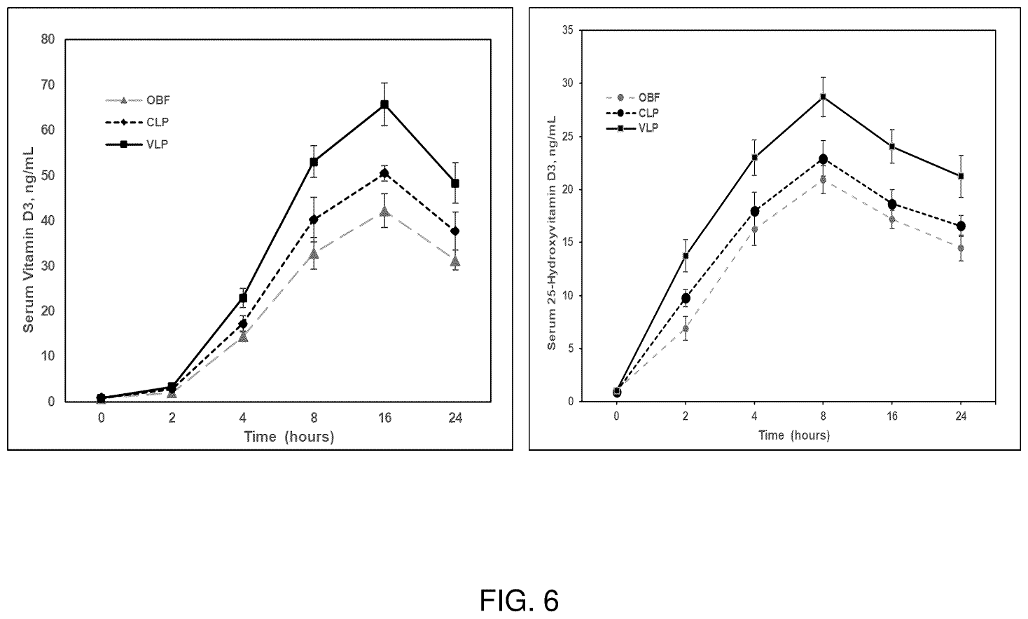

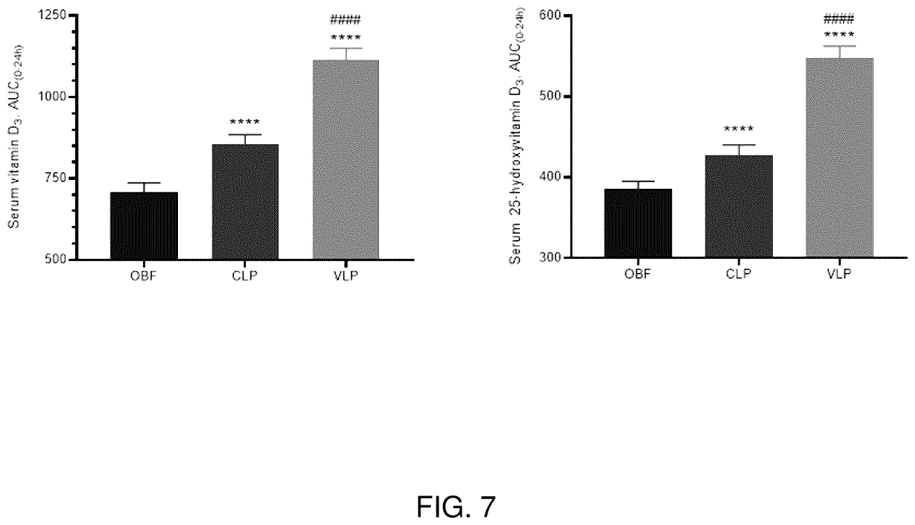

Vitamin D3 and 25-OH vitamin D3 enzyme-linked immunosorbent assay (ELISA) assay kits, compatible with rats, were used to measure serum concentrations. The obtained results are shown in FIG. 6 and FIG. 7.

As demonstrated in the graphs of FIG. 6 and FIG. 7, compared to the oil-based formulation (OBF) and classic liposome formulation (CLP), VeCell liposomes (VLP) enhanced the bioavailability of Vitamin D3 and 25-hydroxy Vitamin D3.

### Comparative Study 2: VeCell Liposomal Vitamin K Vs Oil-Based Formulation

Evaluate the bioavailability of Vecell liposomal Vitamin K (Example 9) compared to traditional oil-based Vitamin K supplement and Unencapsulated Vitamin K in Hydrophilic Matrix using an animal model, i.e., Sprague Dawley rats. The traditional oil-based product comprises vitamin K, olive oil, beeswax, glycerin, purified water, microcrystalline cellulose, and maltodextrin. The Unencapsulated Vitamin K product comprises Vitamin K dispersed in hydrophilic matrix.

The rats were randomly allocated into 3 groups (n=6 each). Baseline blood samples (TO) were collected one-day prior. On the 1^{st} day, one dose of the Vitamin K formulations was administrated to the animals in the morning. Blood withdrawals were performed at times points 1, 2, 4, 6, 8, 16 and 24 hours after oral administration.

The dosing continued on Day 2 and Day 3 after the sample withdrawal. Blood samples were collected daily at 4^{th} and 24^{th} hour after the dosing during this Vitamin K feeding period. The dosing stopped on Day 4, but blood samples were drawn daily from the 4^{th} to 7^{th} day.

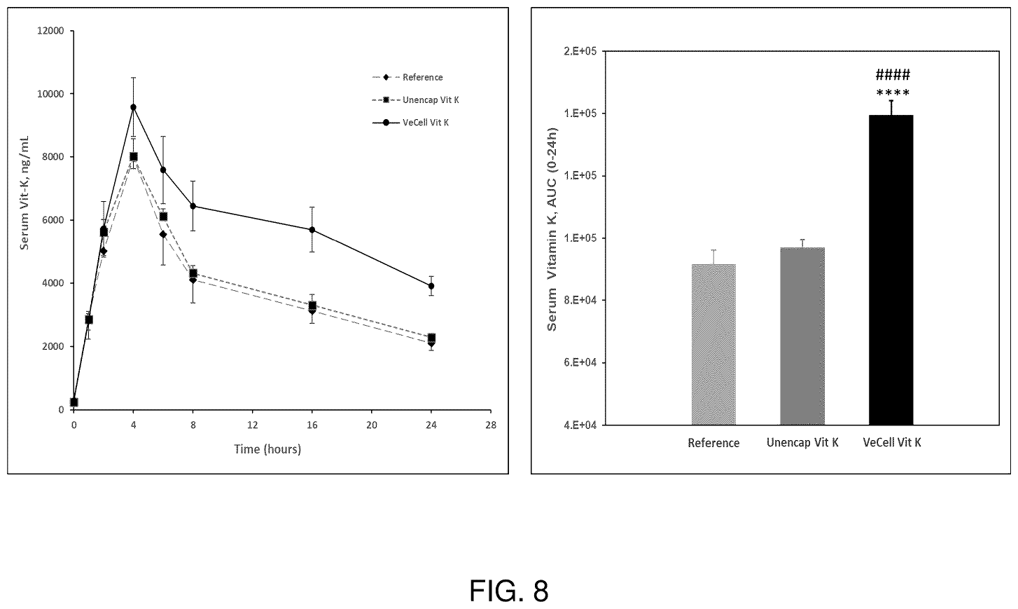

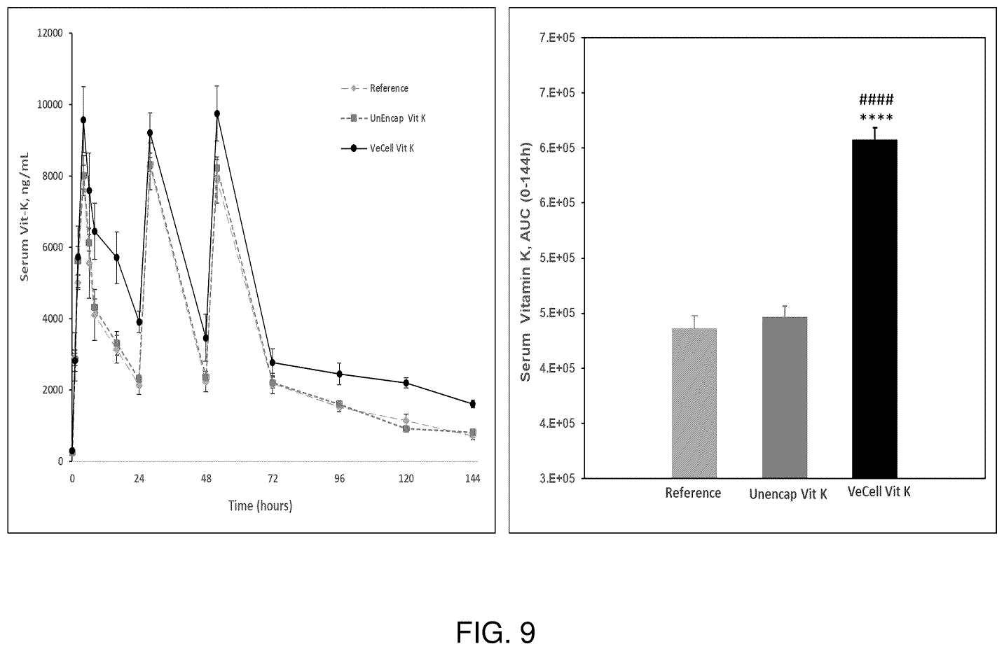

The Vitamin K concentration in sera samples were determined using an HPLC method. The obtained results are shown in FIG. 8 and FIG. 9.

As demonstrated in the graphs, compared to the traditional oil-based formulation and unencapsulated Vitamin K in hydrophilic matrix, VeCell liposomes show superior bioavailability of Vitamin K. Furthermore, the serum Vitamin K level remained higher (>80%) after the feeding stopped, indicating a sustained release profile.

### INCORPORATION BY REFERENCE

All publications, patents, and patent applications mentioned herein are hereby incorporated by reference in their entirety as if each individual publication, patent, or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A lithe liposome composition, comprising:
(i) a phospholipid;
(ii) a lipid bilayer modifier, wherein the lipid bilayer modifier is selected from the group consisting of a surfactant, a solvent, a polysaccharide, and a combination thereof;
(iii) an absorption enhancer, wherein the absorption enhancer is selected from the group consisting of a terpene, a polyphenol, and a derivative thereof, and a combination thereof; and/or
(iv) an active agent.

2. The composition of claim 1,
(i) wherein the phospholipid is selected from the group consisting of a glycerophospholipid, a sphingophospholipid, a derivative thereof, and a combination thereof;
(ii) wherein the phospholipid is selected from the group consisting of a phosphatidylcholine (PC), a phosphatidylinositol (PI), a phosphatidylinositol phosphate (PIP), a phosphatidylinositol bisphosphate (PIP2), a phosphatidylinositol trisphosphate (PIP3), a phosphorylglycerol (PG), a phosphatidic acid (PA), a phosphatidylethanolamine (PE), a phosphatidylserine (PS), a sphingomyelin (SPM), a sphingosylphosphorylethanolamine (SPE), cardiolipin (CL), a derivative thereof, and a combination thereof;
(iii) wherein the phospholipid is selected from the group consisting of a natural phospholipid, a synthetic phospholipid, and a combination thereof, optionally wherein the phospholipid comprises a phosphatidylcholine (PC) or a derivative thereof; and/or
(iv) wherein the phospholipid is present in an amount of about 0.01% w/v to about 50% w/v, optionally wherein the composition comprises:
a phospholipid in an amount of about 0.01% w/v to about 15% w/v;
a phospholipid in an amount of about 0.01% w/v to about 10% w/v;
a phospholipid in an amount of about 0.01% w/v to about 5% w/v;
a phospholipid in an amount of about 0.01% w/v to about 1% w/v;
a phospholipid in an amount of about 0.1% w/v to about 1% w/v;
a phospholipid in an amount of about 0.5% w/v to about 5% w/v;
a phospholipid in an amount of about 5% w/v to about 15% w/v;
a phospholipid in an amount of about 10% w/v to about 15% w/v;
a phospholipid in an amount of about 1% w/v to about 3% w/v;
a phospholipid in an amount of about 2% w/v; or
a phospholipid in an amount of about 1% w/v.

3-5. (canceled)

6. The composition of claim 1,
(i) wherein the lipid bilayer modifier is selected from the group consisting of an anionic surfactant, a cationic surfactant, a zwitterionic surfactant, a non-ionic surfactant, and a combination thereof;
(ii) wherein the lipid bilayer modifier is selected from the group consisting of a polysorbate 20, a polysorbate 60, and a polysorbate 80, a span 20, a span 40, a span 60, a span 80, a bile salt, sodium cholate, cyclodextrin, ethanol, and a combination thereof; and/or
(iii) wherein the lipid bilayer modifier is present in an amount of about 0.01% w/v to about 50% w/v, optionally wherein the composition comprises:
a lipid bilayer modifier in an amount of about 0.01% w/v to about 15% w/v;
a lipid bilayer modifier in an amount of about 0.01% w/v to about 10% w/v;
a lipid bilayer modifier in an amount of about 0.01% w/v to about 5% w/v;
a lipid bilayer modifier in an amount of about 0.01% w/v to about 1% w/v;
a lipid bilayer modifier in an amount of about 0.1% w/v to about 1% w/v;
a lipid bilayer modifier in an amount of about 0.5% w/v to about 5% w/v;
a lipid bilayer modifier in an amount of about 5% w/v to about 15% w/v;
a lipid bilayer modifier in an amount of about 10% w/v to about 15% w/v;
a lipid bilayer modifier in an amount of about 1% w/v to about 3% w/v;
a lipid bilayer modifier in an amount of about 2% w/v; or
a lipid bilayer modifier in an amount of about 1% w/v.

7-8. (canceled)

9. The composition of claim 1,
(i) wherein the absorption enhancer comprises a terpene, optionally wherein the terpene is selected from the group consisting of limonene, camphor, menthol, carvone, terpineol, thujone, a derivative thereof, and a combination thereof, optionally wherein the terpene comprises menthol;
(ii) wherein the absorption enhancer comprises a polyphenol, optionally, wherein the polyphenol comprises a flavonoid, optionally wherein the flavonoid is selected from the group consisting of an isoflavone, a neoflavonoid, a flavone, a flavonol, a flavanone, a flavanonol, a flavanol, a catechin, an anthocyanin, a chalcone, and a derivative thereof, and a combination thereof; or wherein the flavonoid is selected from the group consisting of cyanidin, malvidin, delphinidin, peonidin, phloretin, arbutin, phlioridzin, chalconaringenin, hesperitin, naringin, naringenin, eriodictyol, hesperidin, apigenin, tangeretin, baicalein, rpoifolin, quercetin, dihydroquercetin, myricetin, rutin, morin, kaempferol, genistin, genistein, daidzein, glycitein, daidzin, a derivative thereof, and a combination thereof;
(iii) wherein the absorption enhancer comprises a terpene and a polyphenol;
(iv) wherein the absorption enhancer comprises a flavonoid comprising quercetin, dihydroquercetin, or a combination thereof; and/or
(v) wherein the absorption enhancer is present in an amount of about 0.01% w/v to about 50% w/v, optionally wherein the composition comprises:
an absorption enhancer in an amount of about 0.01% w/v to about 15% w/v;
an absorption enhancer in an amount of about 0.01% w/v to about 10% w/v;
an absorption enhancer in an amount of about 0.01% w/v to about 5% w/v;
an absorption enhancer in an amount of about 0.01% w/v to about 1% w/v;
an absorption enhancer in an amount of about 0.1% w/v to about 1% w/v;
an absorption enhancer in an amount of about 0.5% w/v to about 5% w/v;
an absorption enhancer in an amount of about 5% w/v to about 15% w/v;
an absorption enhancer in an amount of about 10% w/v to about 15% w/v;
an absorption enhancer in an amount of about 1% w/v to about 3% w/v;
an absorption enhancer in an amount of about 2% w/v; or
an absorption enhancer in an amount of about 1% w/v.

10-14. (canceled)

15. The composition of claim 1, which is substantially free of quercetin and/or dihydroquercetin.

16-18. (canceled)

19. The composition of claim 1,
(a) wherein the active agent is characterized by one or more of the following:
(i) low solubility and high permeability;
(ii) high solubility and low permeability;
(iii) low solubility and low permeability;
(iv) low bioavailability;
(v) moderate to severe side effects; and
(iv) instability; and combinations thereof;
(b) wherein the active agent is characterized according to a biopharmaceutical classification system (BCS) as set forth in Table 1, optionally wherein the active agent is selected from the group consisting of
(i) a Class I drug, optionally characterized by an apparent permeability (Papp) of greater than about 10⁻⁵ (cm/sec) and a dose/solubility (Q) of less than or equal to about 0.5;
(ii) a Class II drug, optionally characterized by an Papp of greater than about 10⁻⁵ (cm/sec) and a Q of greater than about 1.0;
(iii) a Class III drug, optionally characterized by an Papp of less than about 2×10⁻⁶ (cm/sec) and a Q of less than or equal to about 0.5; and
(iv) a Class IV drug, optionally characterized by an Papp of less than about 2×10⁻⁶ (cm/sec) and a Q of greater than about 1.0;
(c) wherein the active agent is selected from the group consisting of a vitamin, a curcuminoid, a cannabinoid, a plant alkaloid, quinone, derivatives thereof, salts thereof, and combinations thereof;
(d) wherein the composition comprises at least one cannabinoid selected from the group consisting of cannabidiol (CBD), cannabidiolic acid (CBDA), cannabinol (CBN), cannabinolic acid (CBNA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabivarinic acid (CBVA), tetrahydrocannabivarin (THCV), tetrahydrocannabivarinic acid (THCVA) cannabidivarin (CBDV), cannabidivarinic acid (CBDVA), cannabichromevarin (CBCV), cannabichromevarinic acid (CBCVA), cannabigerovarin (CBGV), cannabigerovarinic acid (CBGVA), salt thereof, derivatives thereof, and combinations thereof;
(e) wherein the composition comprises at least one curcuminoid selected from the group consisting of curcumin, demethoxycurcumin (DMC), bis-demethoxycurcumin (BDMC), salts thereof, derivatives thereof, and combinations thereof;
(f) wherein the composition comprises at least one vitamin selected from the group consisting of vitamin A, vitamin C, vitamin D2 (ergocalciferol), vitamin D3 (cholecalciferol), vitamin E (α-tocopherol), vitamin K, vitamin K1, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folate), vitamin B12 (cyanocobalamin), salts thereof, derivatives thereof, and combinations thereof;
(g) wherein the composition comprises at least one plant alkaloid selected from the group consisting of berberine, salts thereof, derivatives thereof, and combinations thereof; and/or
(h) wherein the composition comprises at least one quinone selected from the group consisting of Coenzyme Q10 (CoQ10), salts thereof, derivatives thereof, and combinations thereof.

20-22. (canceled)

23. The composition of claim 1, wherein the active agent is present in an amount of about 0.01% w/v to about 50% w/v, optionally wherein the composition comprises:
an active agent in an amount of about 0.01% w/v to about 15% w/v;
an active agent in an amount of about 0.01% w/v to about 10% w/v;
an active agent in an amount of about 0.01% w/v to about 5% w/v;
an active agent in an amount of about 0.01% w/v to about 1% w/v;
an active agent in an amount of about 0.1% w/v to about 1% w/v;
an active agent in an amount of about 0.5% w/v to about 5% w/v;
an active agent in an amount of about 5% w/v to about 15% w/v;
an active agent in an amount of about 10% w/v to about 15% w/v;
an active agent in an amount of about 1% w/v to about 3% w/v;
an active agent in an amount of about 2% w/v; or
an active agent in an amount of about 1% w/v.

24. The composition of claim 1, further comprising a sterol or a derivative thereof, optionally wherein the sterol or the derivative thereof comprises a cholesterol or a derivative thereof, optionally wherein the sterol or the derivative thereof is present in an amount of about 0.01% w/v to about 50% w/v, optionally wherein the composition comprises:
a sterol or a derivative thereof in an amount of about 0.01% w/v to about 15% w/v;
a sterol or a derivative thereof in an amount of about 0.01% w/v to about 10% w/v;
a sterol or a derivative thereof in an amount of about 0.01% w/v to about 5% w/v;
a sterol or a derivative thereof in an amount of about 0.01% w/v to about 1% w/v;
a sterol or a derivative thereof in an amount of about 0.1% w/v to about 1% w/v;
a sterol or a derivative thereof in an amount of about 0.5% w/v to about 5% w/v;
a sterol or a derivative thereof in an amount of about 5% w/v to about 15% w/v;
a sterol or a derivative thereof in an amount of about 10% w/v to about 15% w/v;
a sterol or a derivative thereof in an amount of about 1% w/v to about 3% w/v;
a sterol or a derivative thereof in an amount of about 2% w/v; or
a sterol or a derivative thereof in an amount of about 1% w/v.

25. The composition of claim 1, which is substantially free of a cholesterol or a derivative thereof.

26. The composition of claim 1, comprising:
(i) a phospholipid,
an active agent,
a cholesterol or a derivative thereof,
a lipid bilayer modifier and/or
a hydrophilic matrix;
(ii) a phospholipid,
an active agent,
a cholesterol or a derivative thereof,
a lipid bilayer modifier,
an absorption enhancer, and/or
a hydrophilic matrix;
(iii) a phospholipid,
an active agent,
a cholesterol or a derivative thereof,
a lipid bilayer modifier, optionally comprising a surfactant,
an absorption enhancer,
an additional lipid bilayer modifier, optionally comprising a solvent, and/or
a hydrophilic matrix,
(iv) a phospholipid,
an active agent,
a lipid bilayer modifier, and/or
a hydrophilic matrix; or
(v) a phospholipid,
an active agent,
a lipid bilayer modifier,
an absorption enhancer, and/or
a hydrophilic matrix.

27. The composition of claim 1, comprising:
(a) a phospholipid comprising a phosphatidylcholine (PC) or a derivative thereof;
an active agent comprising a cannabidiol (CBD) or a derivative thereof;
a cholesterol or a derivative thereof;
a lipid bilayer modifier comprising span 60 or a derivative thereof; and/or
a hydrophilic matrix; or
(b) a phospholipid comprising a phosphatidylcholine (PC) or a derivative thereof;
an active agent comprising a cannabidiol (CBD) or a derivative thereof;
a cholesterol or a derivative thereof;
a lipid bilayer modifier comprising a polysorbate 80 or a derivative thereof;
an absorption enhancer comprising a quercetin or a derivative thereof; and/or
a hydrophilic matrix.

28. (canceled)

29. The composition of claim 1, comprising:
a phospholipid comprising a phosphatidylcholine (PC) or a derivative thereof;
an active agent comprising a berberine hydrochloride or a derivative thereof;
a lipid bilayer modifier comprising a bile acid, a bile salt, or a derivative thereof; and/or
a hydrophilic matrix.

30. The composition of claim 1, comprising:
(a) a phospholipid comprising a phosphatidylcholine (PC) or a derivative thereof;
an active agent comprising a Vitamin D3 or a derivative thereof;
a cholesterol or a derivative thereof;
a lipid bilayer modifier comprising a sodium cholate or a derivative thereof; and/or
a hydrophilic matrix;
(b) a phospholipid comprising a phosphatidylcholine (PC) or a derivative thereof;
an active agent comprising a Vitamin D3 or a derivative thereof;
a cholesterol or a derivative thereof;
an absorption enhancer comprising a dihydroquercetin or a derivative thereof;
a lipid bilayer modifier comprising sodium cholate; and/or
a hydrophilic matrix;
(c) a phospholipid comprising a phosphatidylcholine (PC) or a derivative thereof;
an active agent comprising a Vitamin D3 or a derivative thereof;
a cholesterol or a derivative thereof;
an absorption enhancer comprising menthol;
a lipid bilayer modifier comprising sodium cholate or a derivative thereof; and/or
a hydrophilic matrix; or
(d) a phospholipid comprising a phosphatidylcholine (PC) or a derivative thereof;
an active agent comprising a Vitamin D3 or a derivative thereof;
a cholesterol or a derivative thereof;
a lipid bilayer modifier comprising a sodium cholate or a derivative thereof; and/or
a hydrophilic matrix.

31-33. (canceled)

34. The composition of claim 1, comprising:
a phospholipid comprising a phosphatidylcholine (PC) or a derivative thereof;
an active agent comprising a cannabidiol (CBD) or a derivative thereof;
a cholesterol or a derivative thereof;
a lipid bilayer modifier comprising a surfactant, optionally wherein the surfactant comprises span 80 or a derivative thereof;
an absorption enhancer comprising a dihydroquercetin or a derivative thereof;
a lipid bilayer modifier comprising a solvent, optionally wherein the solvent comprises methanol or a derivative thereof; and/or
a hydrophilic matrix.

35. The composition of claim 1, comprising:
a phospholipid comprising a phosphatidylcholine (PC) or a derivative thereof;
an active agent comprising a Vitamin K1 or a derivative thereof;
a lipid bilayer modifier comprising a bile acid, a bile salt, or a derivative thereof;
an absorption enhancer comprising a quercetin or a derivative thereof; and/or
a hydrophilic matrix.

36. The composition of claim 1, comprising:
(a) a phospholipid comprising a phosphatidylcholine (PC) or a derivative thereof;
an active agent comprising a curcuminoid or a derivative thereof;
a lipid bilayer modifier comprising a hydroxypropyl-β-cyclodextrin (HBC) or a derivative thereof; and/or
a hydrophilic matrix; or
(b) a phospholipid comprising a phosphatidylcholine (PC) or a derivative thereof;
an active agent comprising a curcuminoid or a derivative thereof;
a lipid bilayer modifier comprising a bile acid, a bile salt, or a derivative thereof;
an absorption enhancer comprising a quercetin or a derivative thereof; and/or
a hydrophilic matrix.

37. (canceled)

38. The composition of claim 1, comprising a vesicle morphology characterized by a lipid bilayer structure.

39. The composition of claim 1, wherein the composition is an oral composition.

40. The composition of claim 1,
(i) which is characterized by improved solubility of an active agent as compared to a reference composition, optionally wherein the reference composition comprises an oil-based composition, a micellized composition, and/or a conventional liposome, optionally wherein the solubility is tested using a buffer selected from the group consisting of phosphate-buffered saline (PBS), simulated gastric fluid (SGF), fasted state simulated intestinal fluid (FaSSIF), and fed state simulated intestinal fluid (FeSSIF);
(ii) which is characterized by improved passive, transcellular permeability and/or permeability across intestinal epithelia as compared to a reference composition, optionally wherein the reference composition comprises an oil-based composition, a micellized composition, and/or a conventional liposome, optionally wherein the permeability is tested using a buffer selected from the group consisting of phosphate-buffered saline (PBS), simulated gastric fluid (SGF), fasted state simulated intestinal fluid (FaSSIF), and fed state simulated intestinal fluid (FeSSIF);
(iii) which is characterized by improved stability in the gastrointestinal tract as compared to a reference composition, optionally wherein the reference composition comprises an oil-based composition, a micellized composition, and/or a conventional liposome;
(iv) which is characterized by improved oral absorption of the active agent as compared to a reference composition, optionally wherein the reference composition comprises an oil-based composition, a micellized composition, and/or a conventional liposome;
(v) which is characterized by improved oral bioavailability of the active agent as compared to a reference composition, optionally wherein the reference composition comprises an oil-based composition, a micellized composition, and/or a conventional liposome; and/or
(vi) which is characterized by improved bio-variability as compared to a reference composition, optionally wherein the reference composition comprises an oil-based composition, a micellized composition, and/or a conventional liposome.

41-45. (canceled)

46. A pharmaceutical composition comprising the lithe liposome composition of claim 1 and optionally a pharmaceutically acceptable carrier.

47. The pharmaceutical composition of claim 46, wherein the pharmaceutical composition is an oral pharmaceutical composition.

48. A method of preparing a lithe liposome composition, comprising:
(i) dissolving a phospholipid in an organic solvent to form a lipid mixture;
(ii) optionally contacting the lipid mixture with a lipid bilayer modifier, an absorption enhancer, or a combination thereof;
(iii) optionally contacting the lipid mixture with an active agent, optionally one or more active agent(s);
(iv) drying the lipid mixture to remove the organic solvent to form a dried lipid thin film;
(v) hydrating the dried lipid thin film with a hydrophilic solution (e.g., an aqueous solution) to form a liposomal suspension;
(vi) optionally contacting the liposomal suspension with a lipid bilayer modifier, an absorption enhancer, or a combination thereof;
(vii) optionally contacting the liposomal suspension with one or more hydrophilic active agent(s); and
(viii) optionally agitating, stirring, and/or sonicating the liposomal suspension.

49. A method of enhancing the stability of an active agent in the gastrointestinal tract of a subject in need thereof; improving the permeability of an active agent across the intestinal epithelia of a subject in need thereof; improving the oral absorption, the oral bioavailability, and/or the bio-variability of an active agent in a subject in need thereof; or treating and/or preventing a disease or condition in a subject in need thereof, comprising administering the lithe liposome composition of claim 1 to the subject, optionally by oral administration.

50-53. (canceled)
